(19) United States
(12) Patent Application Publication
(10) Pub. No.: US 2025/0171775 A1
(43) Pub. Date: May 29, 2025

(54) PROMOTERS FOR SPECIFIC EXPRESSION OF GENES IN CONE PHOTORECEPTORS

(71) Applicant: INSTITUTE OF MOLECULAR AND CLINICAL OPHTHALMOLOGY BASEL (IOB), Basel (CH)

(72) Inventors: Botond Miklós Roska, Oberwil (CH); Hendrik P.N. Scholl, Binningen (CH); Josephine JÜTTNER, Allschwil (CH); Stefan Spirig, Zurich (CH)

(21) Appl. No.: 19/044,002

(22) Filed: Feb. 3, 2025

Related U.S. Application Data
(63) Continuation of application No. PCT/IB2023/058052, filed on Aug. 9, 2023.
(60) Provisional application No. 63/397,183, filed on Aug. 11, 2022.
(60) Provisional application No. 63/399,824, filed on Aug. 22, 2022.

(51) Int. Cl. C12N 15/11; C12N 15/86

(52) U.S. Cl. CPC C12N 15/11; C12N 15/86; C12N 2750/14143; C12N 2820/007; C12N 2830/48; C12N 2830/50

(57) ABSTRACT

Disclosed herein are synthetic cone-specific promoters. The promoters can be used in nucleic acids, vectors, or host cells to drive expression of a heterologous polypeptide or RNA in retinal cells, particularly cones.

### RELATED APPLICATIONS

This application claims the benefit of U.S. Provisional Patent Application No. 63/397,183 filed Aug. 11, 2022, and U.S. Provisional Patent Application No. 63/399,824, filed Aug. 22, 2022, the entire contents of each of which is incorporated herein by reference.

### BACKGROUND

Blindness disables millions of people worldwide and is a major health problem. One common cause of blindness is the dysfunction of the retina and in particular of the photoreceptor cells. Most common forms of retinal blindness are Retinitis Pigmentosa (RP) and macular degeneration (AMD) inter alia causing a degeneration of photoreceptors cells and the consequent loss of light sensitivity. There is a need to be able to obviate the problems associated with such degeneration of photoreceptors or loss in their light sensitivity. This can preferably be effected by the expression of therapeutic molecules, in particular, polypeptides, kinines, trophic factors, channels, opsins or receptors, or of nucleic acid molecules, specifically in the cone photoreceptor cells.

For expression, recombinant genes or exogenous or heterologous genes are usually transfected into the target cells, cell populations or tissues, typically as cDNA constructs in the context of an active expression cassette to allow transcription of the heterologous gene. The DNA construct is recognized by the cellular transcription machinery in a process that involves the activity of many trans-acting transcription factors (TF) at cis-regulatory elements, including enhancers, silencers, insulators and promoters, herein all generally referred to as “regulatory elements”. Gene promoters are involved in all of these levels of regulation, serving as the determinant in gene transcription by integrating the influences of the DNA sequence, transcription factor binding and epigenetic features. They determine the strength of, for example, transgene expression as well as in which cell type or types said transgene will be expressed.

Common promoters used for driving heterologous gene expression in mammalian cells are the human and mouse cytomegalovirus (CMV) major immediate early promoter. They confer a strong expression and have proved robust in several cell types. Other viral promoters such as the SV40 immediate early promoter and the Rous Sarcoma Virus (RSV) long-terminal-repeat (LTR) promoter are also used frequently in expression cassettes. Instead of viral promoters, cellular promoters can also be used. Among known promoters are those from house-keeping genes that encode abundantly transcribed cellular transcripts, such as beta-actin, elongation factor 1-alpha (EF-1alpha), or ubiquitin. Compared to viral promoters, eukaryotic gene expression is more complex and requires a precise coordination of many different factors.

Aspects of concern regarding the use of endogenous regulatory elements for transgene expression include the generation of stable mRNA, and that expression can take place in the native environment of the host cell where trans-acting transcription factors are provided accordingly. Since expression of eukaryotic genes is controlled by a complex machinery of cis- and trans-acting regulatory elements, most cellular promoters suffer from a lack of extensive functional characterization. Parts of the eukaryotic promoter are usually located immediately upstream of its transcribed sequence and serve as the point of transcriptional initiation. The core promoter immediately surrounds the transcription start site (TSS) which is sufficient to be recognized by the transcription machinery. The proximal promoter comprises the region upstream of the core promoter and contains the TSS and other sequence features required for transcriptional regulation. Transcription factors act sequence-specific by binding to regulatory motifs in the promoter and enhancer sequence.

Some promoters can act in a cell specific manner and can be used to express a transgene in cells of a specific type or in cells of a particular subset. However, the development of promoters for retinal cells, such as cone cells, has been hampered by relatively low specificity (i.e., the promoters can also drive relatively high expression in other cell types, such as rods).

Accordingly, there is a need for promoters with higher specificity for expression in cone cells.

### SUMMARY

This disclosure relates to isolated nucleic acids comprising a cone-specific promoter. The promoter comprises a first nucleotide sequence of at least about 150 nucleotides which can have at least 70% identity to a sequence of equal length from the sequence of SEQ ID NO: 1; and a second nucleotide sequence of at least about 260 nucleotides having at least 70% identity to a sequence of equal length from the sequence of SEQ ID NO: 2. In embodiments, the promoter comprises a first nucleotide sequence of at least about 150 nucleotides which can have at least 70% identity to a sequence of equal length from the sequence of SEQ ID NO: 1; and a second nucleotide sequence of at least about 370 nucleotides having at least 70% identity to a sequence of equal length from the sequence of SEQ ID NO: 2.

The first nucleotide sequence can have at least 70% identity to a sequence of equal length from the 3′ end of the sequence of SEQ ID NO: 1. The first nucleotide sequence can have at least 90% identity to a sequence of equal length from the 3′ end of the sequence of SEQ ID NO: 1; the second nucleotide sequence can have at least 90% identity to a sequence of equal length from the 3′ end or the 5′ end of the sequence of SEQ ID NO: 2, or both. The first nucleotide sequence can comprise from about 150 nucleotides to about 395 nucleotides from the 3′ end of the sequence of SEQ ID NO: 1. The first nucleotide sequence can comprise SEQ ID NO: 12, SEQ ID NO: 13, SEQ ID NO: 14, or SEQ ID NO: 15.

The second nucleotide sequence can comprise from about 260 nucleotides to about 895 nucleotides from the 3′ end of SEQ ID NO: 2 or about 895 nucleotides from the 5′ end of SEQ ID NO: 2. The second nucleotide sequence can comprise SEQ ID NO: 21, SEQ ID NO: 22, SEQ ID NO: 23, SEQ ID NO: 24, SEQ ID NO: 25, SEQ ID NO: 26, or SEQ ID NO: 30. The second nucleotide sequence can comprise SEQ ID NO:39.

The isolated nucleic acid can comprise from 5′ to 3′ the first nucleotide sequence and the second nucleotide sequence with no sequences therebetween. The isolated nucleic acid can comprise from 5′ to 3′ the second nucleotide sequence and the first nucleotide sequence with no sequences therebetween. The isolated nucleic acid can comprise two or more first nucleotide sequences, two or more second nucleotide sequences, or both.

The isolated nucleic acid can further comprise a nucleotide sequence encoding a heterologous polypeptide or a heterologous RNA, wherein the promoter and the nucleotide sequence encoding the polypeptide are operably linked.

The isolated nucleic acid can further comprise a regulatory element; wherein the promoter, the regulatory element, and the nucleotide sequence encoding the heterologous polypeptide or heterologous RNA, if present, are operably linked.

The regulatory element can be a Woodchuck Hepatitis Virus Posttranscriptional Regulatory element (WPRE).

The isolated nucleic acid can further comprise a nucleotide sequence encoding a polyadenylation signal (Poly A) that is 3′ of the nucleotide sequence encoding the regulatory element; wherein the nucleotide sequence encoding the Poly A and the nucleotide sequence encoding the regulatory element are operably linked.

The isolated nucleic acid can comprise a first AAV inverted terminal repeat sequence (ITR) that is 5′ of the promoter and a second AAV ITR that is 3′ of the regulatory element and preferably 3′ of the Poly A signal.

The isolated nucleic acid can further comprise a nucleotide sequence encoding an AAV inverted terminal repeat sequence (ITR).

This disclosure also relates to an isolated nucleic acid, comprising a cone-specific promoter, the promoter comprising a first nucleotide sequence of at least about 150 nucleotides and no more than 499 nucleotides which can have at least 70% identity to a sequence of equal length from the sequence of SEQ ID NO: 1. For example, the cone-specific promoter can be SEQ ID NO: 12, SEQ ID NO: 13, SEQ ID NO: 14, SEQ ID NO: 15 or a sequence that has at least 70% identity to any of the foregoing. For example the cone-specific promoter can be SEQ ID NO: 40 or a sequence that has at least 70% identity to any of the foregoing.

This disclosure further relates to a vector comprising any isolated nucleic acid as described herein.

The vector can further comprise a viral capsid.

The viral capsid can be an adeno-associated viral vector (AAV) capsid selected from the group consisting of AAV8-BP2 capsid, AAV-PHP.B capsid, AAV-PHP.eB capsid, AAV5 capsid, or AAV-NHP26 capsid.

Disclosed herein is a host cell comprising a vector as described herein.

The disclosure also relates to kits comprising an isolated nucleic acid, a vector a host cell, or two or more thereof.

This disclosure also relates to a method of expressing a heterologous polypeptide or a heterologous RNA in a retinal organoid, a retina explant, or a mammalian subject. The method can comprise administering to the retinal organoid, the retina explant, or the mammalian subject an isolated nucleic acid, a vector, a host cell, or two or more thereof.

### BRIEF DESCRIPTION OF THE DRAWINGS

FIG. 1 schematically depicts nucleic acid constructs in which ProA7 and truncated ProA7 promoters were studied in Example 1.

FIG. 2 shows maximum intensity Z-projections of whole organoid live images of human retinal organoids transduced with AAVs, delivering constructs comprising ProA7 or truncated ProA7 promoters as indicated.

FIGS. 3A-3C shows quantification plots of whole organoid live imaging for constructs comprising ProA7 or truncated ProA7 promoters as indicated. FIG. 3A shows the number of GFP expressing cells detected per well. FIG. 3B shows the calculated density of GFP expressing cells per mm². FIG. 3C shows the mean GFP intensity in the detected cells in relative fluorescent units (RFU). Experiment was done in triplicate, except for ProA7 (6 replicates).

FIG. 4 shows spinning disk confocal microscope images of cross-sections of human retinal organoids infected with AAV-ProA7-GFP (top row), AAV-ProA7 5′3-GFP (middle row), or AAV-ProA7 5′6-GFP (bottom row). Left column: GFP (green). Middle column, immunostaining with cone marker CAR (magenta). Right column: GFP and cone marker (signal overlap visible in white).

FIG. 5 schematically depicts nucleic acids for AAV generation: pAAV-{ProA7/Pro572/Pro573/Pro572.2/Pro573.2}-GFP-WPRE-hGHpA.

FIGS. 6A-6C presents quantification plots of whole organoid live imaging for constructs comprising ProA7 or truncated ProA7/rod-specific promoters as indicated. FIG. 6A shows the number of GFP expressing cells detected per well. FIG. 6B shows the calculated density of GFP expressing cells per mm2. FIG. 6C shows the mean GFP intensity in the detected cells in relative fluorescent units (RFU). Experiment was done in triplicate.

FIG. 7 shows spinning disk confocal microscope images of cross-sections of human retinal organoids infected with AAV-Pro572-GFP and AAV-Pro573-GFP, respectively. Left: GFP (green). Middle-left, immunostaining with cone marker CAR (magenta). Middle-right: GFP and cone marker (signal overlap visible in white). Right: GFP and cone marker and nuclear stain (Hoechst, white).

FIGS. 8A-8B are graphs showing that the transgene expression of the hybrid promoter variants remain specific for cone photoreceptors. FIG. 8A shows a quantification plot for expression in cross-sections of human retinal organoids infected by AAV-{Pro572/Pro572.2/Pro573/Pro573.2}-GFP and AAV-ProA7-GFP. Quantification of GFP+ cell density as a percentage of cone photoreceptor density; values are the mean±s.e.m. from n=10 confocal images. FIG. 8B shows quantification of AAV-targeting specificity shown as a percentage of the cones (black, Cones (CAR+)) and minor cell types (gray, Others) cell types or classes among cells expressing GFP in organoids infected by AAV-Pro572.2-GFP and AAV-Pro573.2-GFP, respectively.

FIGS. 9A-9C are graphs showing the results of a capsid screen using AAV5 and AAVPHP.eB capsids and promoters driving expression of an EGFP transgene following transduction of human retina explants. Vectors tested were: AAV5-Pro572.2-EGFP-WPRE, AAVPHP.eB-Pro572.2-EGFP-WPRE, AAV5-Pro573.2-EGFP-WPRE, AAVPHP.eB-Pro573.3-EGFP-WPRE, AAV5-ProSC-EGFP-WPRE, and AAV5-ProA7-EGFP-WPRE. FIG. 9A and FIG. 9B show quantification of the percentage of total rod and cone photoreceptors expressing GFP under the control of hybrid promoters Pro572.2 or Pro573.2 or cone specific promoters ProSC or ProA7, with a high efficacy (more than 95%) for targeting cone photoreceptors for promoters Pro572.2, Pro573.2, and ProSC in human retina explants. FIG. 9C shows a 3-fold higher mean intensity of GFP signal for human retina explants treated with AAV5-Pro573.2-EGFP-WPRE than those treated with AAV5-ProSC-EGFP-WPRE.

FIG. 10. shows a quantification plot of whole human retinal organoid live imaging of GFP expressing cells following transduction with AAV5 and AAVPHP.eB vectors that contain varying numbers of promoter copies at a dose of 1E10 and 1E11 viral genomes (v.g) per well. Vectors tested were: AAVPHP.eB-ProA7-EGFP-WPRE, AAVPHP.eB-ProSC-EGFP-WPRE, AAVPHP.eB-2×ProSC-EGFP-WPRE, AAVPHP.eB-3×ProSC-EGFP-WPRE, AAVPHP.eB-4×ProSC-EGFP-WPRE, AAV5-ProA330-EGFP-WPRE, AAVPHP.eB-2×min330-EGFP-WPRE, AAVPhP.eB-3×min330-EGFP-WPRE, AAVPHP.eB-4×min330-EGFP-WPRE, AAVPHP.eB-330-3del1del5-EGFP-WPRE, and AAVPHP.eB-3del1del6-EGFP-WPRE. Negative controls include no AAV (Ctrl-noAAV) and AAV5-noPro-EGFP-WPRE (Ctrl-noP).

FIGS. 11A-11B are graphs showing AAVPHP.eB-4×ProSC-EGFP-WPRE which includes a 4× multimerized ProSC promoter remains specific for cone photoreceptors. FIG. 11A shows a quantification plot for expression in whole mounts of cultured human retina explants infected with AAVPHP.eB-ProA7-EGFP-WPRE and AAVPHP.eB-4×ProSC-EGFP-WPRE. Quantification of GFP+ cell density as a percentage of cone photoreceptor density. FIG. 11B shows a quantification plot of AAV-targeting specificity shown as percentage of major (circle Cones) and minor (square Others) cell types or classes among cells expressing GFP in human retina infected by AAVPHP.eB-ProA7-EGFP-WPRE and AAVPHP.eB-4×ProSC-EGFP-WPRE, respectively.

FIG. 12 shows spinning disk confocal microscope images of cross-sections of human retinal organoids transduced with AAVPHP.eB-ProSC-EGFP-WPRE and AAVPHP.eB-4×ProSC-EGFP-WPRE. Left: GFP. Middle-left, immunostaining with cone marker CAR. Middle-right, immunostaining with a rod marker NRL, GFP, and cone marker. Right: GFP and cone marker and nuclear stain.

### DETAILED DESCRIPTION

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it is intended that the particular value forms another embodiment.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “subject” as used herein refers to any animal, such as any mammal, including but not limited to, humans, non-human primates, rodents, mammals commonly kept as pets (e.g., dogs and cats, among others), livestock (e.g., cattle, sheep, goats, pigs, horses, and camels, among others) and the like. In some embodiments, the mammal is a mouse. In some embodiments, the mammal is a human.

The term “cone-specific” as used herein describes a promoter that has promoter activity in human cone photoreceptors that is at least about 90% selective for human cone cells over human rod cells. For example, the promoter can have at least about 91% selectivity, at least about 92% selectivity, at least about 93% selectivity, at least about 94% selectivity, at least about 95% selectivity, at least about 96% selectivity, at least about 97% selectivity, at least about 98% selectivity, at least about 99% selectivity, or 100% selectivity for cone cells over rod cells. The selectivity of a promoter in human cone photoreceptors relative to other cells, such as human rod photoreceptors, can be determined using suitable methods that are well-known to a person of ordinary skill in the art. In one preferred method, a retinal organoid or retinal tissue containing both cone and rod cells is transduced with a nucleic acid construct containing the promoter operably linked to a nucleic acid sequence encoding a reporter molecule, such as tdTomato, enhanced yellow fluorescent protein (EYFP), Citrine, green fluorescent protein (GFP), cyan fluorescent protein, red fluorescent protein, or a functional variant thereof; the organoids are sectioned and immunostained to identify cone cells and/or rod cells using a cell-type-specific antibody (e.g., anti-human cone-arrestin for cone cells, anti-human rhodopsin for rod cells); promoter activity is assessed by counting cells expressing the reporter molecule; and fractions of cells expressing the reporter that are co-labeled with each cell-type-specific antibody are determined. Cone-specificity is calculated as the fraction or percentage of total cells expressing the reporter that are cone cells.

The inventors have observed that the cone-specific synthetic promoters disclosed herein are cone-specific in humans and at least one species of non-human primate (macaques). The promoters may have lower selectivity for cone cells of non-primate mammals.

The term “rod-specific” as used herein describes a promoter that has promoter activity in human rod photoreceptors that is at least about 90% selective for human rod cells over human cone cells. For example, the promoter can have at least about 91% selectivity, at least about 92% selectivity, at least about 93% selectivity, at least about 94% selectivity, at least about 95% selectivity, at least about 96% selectivity, at least about 97% selectivity, at least about 98% selectivity, at least about 99% selectivity, or 100% selectivity for rod cells over cone cells. The selectivity of a promoter in human rod photoreceptors relative to other cells, such as human cone photoreceptors, can be determined using suitable methods that are well-known to a person of ordinary skill in the art. In one preferred method, a retinal organoid or retinal tissue containing both rod and cone cells is transduced with a nucleic acid construct containing the promoter operably linked to a nucleic acid sequence encoding a reporter molecule, such as tdTomato, enhanced yellow fluorescent protein (EYFP), Citrine, green fluorescent protein (GFP), cyan fluorescent protein, red fluorescent protein, or a functional variant thereof; the organoids are sectioned and immunostained to identify rod cells and/or cone cells by using a cell-type-specific antibody (e.g., anti-human cone-arrestin for cone cells, anti-human rhodopsin for rod cells); promoter activity is assessed by counting cells expressing the reporter molecule; and fractions of cells expressing the reporter that are co-labeled with each cell-type-specific antibody are determined. Rod-specificity is calculated as the fraction or percentage of total cells expressing the reporter that are rod cells.

The level of promoter activity in a cell type of interest can be determined by transducing a suitable population of cells (e.g. a human retinal organoid) with a nucleic acid construct containing the promoter operably linked to a nucleic acid sequence encoding a reporter molecule, such as tdTomato, enhanced yellow fluorescent protein (EYFP), Citrine, green fluorescent protein (GFP), cyan fluorescent protein, red fluorescent protein, or a functional variant thereof; the organoids are sectioned and immunostained to identify cells of the cell type of interest by using a cell-type-specific antibody (e.g., anti-human cone-arrestin for cone cells, anti-human rhodopsin for rod cells); counting the number of cells of the cell type of interest; and counting the number of cells of the cell type of interest that express the reporter molecule. Promoter activity in cells of the cell type of interest is calculated as the fraction or percentage of cells of the cell type of interest expressing the reporter molecule.

Additional description of the methods and guidance for the practice of the methods are provided herein.

### A. Promoters

This disclosure relates to synthetic promoters that are cone-specific and can drive expression of a desired nucleic acid (e.g., a transgene) in cone photoreceptor cells. The synthetic promoters can include a first component (a first nucleotide sequence) that is derived from a cone-specific promoter, such as (SEQ ID NO:1), and a second component (a second nucleotide sequence) that is derived from a rod-specific promoter, such as SEQ ID NO:2, with the first and second components operably linked. The inventors have surprisingly discovered that such synthetic promoters are unexpectedly cone-specific, and have augmented promoter activity in human cone cells relative to the cone-specific promoter from which the first component (first nucleotide sequence) is derived (e.g., SEQ ID NO:1).

The cone-specific synthetic promoters disclosed herein can comprise an active fragment of the cone-specific promoter of SEQ ID NO: 1, with the proviso that the synthetic promoter of this disclosure is not SEQ ID NO:1. Such synthetic promoters do not include a nucleotide sequence derived from a rod-specific promoter, such as SEQ ID NO: 2. The inventors unexpectedly determined that such cone-specific synthetic promoters can have augmented promoter activity in human cone cells in comparison to SEQ ID NO: 1.

The synthetic promoters of this disclosure include a first nucleotide sequence that has cone-specific promoter activity. Specific examples of the first nucleotide sequence include SEQ ID NO: 1 and sequences of at least about 150 nucleotides which have at least 70% identity to SEQ ID NO: 1 over the length of the first nucleotide sequence. The first nucleotide sequence is described in more detail herein. The synthetic promoters can consist essentially of the first nucleotide sequence, i.e., can comprise the first nucleotide sequence and omit a second nucleotide sequence as described herein.

The synthetic promoters can comprise a first nucleotide sequence, as described here, and further comprise a second nucleotide sequence, as described herein, that is operably linked to the first nucleotide sequence. The operably linked first nucleotide sequence and second nucleotide sequence function as a single promoter for expression in cone photoreceptors. Promoters comprising both the first nucleotide sequence and the second nucleotide sequence can be referred to herein as hybrid promoters.

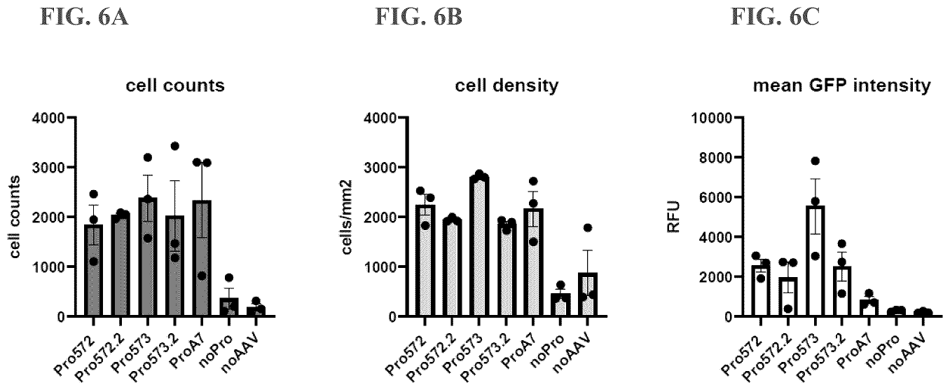

The first and second nucleotide sequences are typically obtained or derived from distinct cone-specific and rod-specific promoters, respectively. As described and exemplified herein, the inventors discovered that such hybrid promoters surprisingly have cone-specific activity, and can result in high levels of expression in cone photoreceptor cells in comparison to the cone-specific promoter from which the first nucleotide sequence is derived. As shown herein, when the first polynucleotide is the cone-specific promoter of SEQ ID NO: 1 or a functional sequence fragment from the 3′ region of SEQ ID NO:1, and the second nucleotide sequence is the rod-specific promoter (SEQ ID NO:2) or a functional sequence fragment thereof, the hybrid promoter was cone-specific and drove higher expression of a reporter (GFP) in cone cells than did SEQ ID NO:1 alone. See, e.g., FIG. 6. Accordingly, this disclosure relates to synthetic promoters, including but not limited to hybrid promoters, to nucleic acids that include the synthetic promoters, and to methods of using such nucleic acids.

### a. First Nucleotide Sequence

The nucleic acids disclosed herein can comprise a synthetic promoter comprising a first nucleotide sequence of at least about 150 nucleotides which has at least 70% identity to SEQ ID NO: 1 over the length of the first nucleotide sequence.

The ProA7 promoter (SEQ ID NO: 1) is known to be useful for driving expression of desired genes in retina of a variety of species. ProA7 is cone-specific, and does not drive substantial expression in other retinal cells. See, e.g., Jüttner et al. Nature Neuroscience 22, 1345-1356 (2019).

As such, the first nucleotide sequence has promoter activity that is specific for cone cells, and can drive expression in human cone cells, for example, cone cells that are dormant and no longer responsive to light. The first nucleotide sequence can have promoter activity in human cone cells, for example cone cells of the retina in vivo, in retinal explants or retinal organoids. In some instances, the first nucleotide sequence does not have substantial promoter activity in other cells of the human retina, such as rod cells (e.g., less than about 10% of promoter activity is seen in non-cone cells).

The ProA7 promoter sequence of SEQ ID NO: 1 has been described in International Publication No. WO2017046084. The first nucleotide sequence may comprise a nucleic acid sequence having at least 70% sequence identity to SEQ ID NO: 1. The first sequence can have a nucleic acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or greater sequence identity to SEQ ID NO: 1 over the length of the first nucleotide sequence.

If desired, the first nucleotide sequence can be a variant of SEQ ID NO: 1 that retains cone-specific promoter activity, either alone or in combination with the optional second nucleotide sequence. The first nucleotide sequence may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, compared to SEQ ID NO: 1, or in the case of a functional fragment of ProA7, compared to the corresponding portion of SEQ ID NO:1.

The promoter can comprise a first nucleotide sequence comprising a sequence of at least 150 nucleotides which has at least 70% identity to SEQ ID NO: 1, preferably the 3′ end of SEQ ID NO:1. As shown herein, such fragments of SEQ ID NO:1 retain cone-specific promoter activity.

The first nucleotide sequence can comprise a sequence having at least 70% identity to a sequence of equal length from the 3′ end of SEQ ID NO: 1. A first sequence can be considered to be a 5′ truncation of SEQ ID NO: 1, because nucleotides from the 5′ end of SEQ ID NO: 1 have been removed to arrive at the first sequence.

A first nucleotide sequence can be a 5′ truncation of SEQ ID NO: 1 and can contain fewer than the 500 nt of ProA7, due to deletion of nucleotides at the 5′ end. For example, the first nucleotide sequence can be the same as nucleotides 386-500 of SEQ ID NO:1 or can be the same as nucleotides 2-500 of SEQ ID NO:1. Such a first nucleotide sequence can comprise any number of nucleotides from 150 to 499 (i.e., one less than the length of SEQ ID NO: 1).

For example, the first nucleotide sequence can comprise about 150 nucleotides (nt), about 151 nt, about 152 nt, about 153 nt, about 154 nt, about 155 nt, about 156 nt, about 157 nt, about 158 nt, about 159 nt, about 160 nt, about 161 nt, about 162 nt, about 163 nt, about 164 nt, about 165 nt, about 166 nt, about 167 nt, about 168 nt, about 169 nt, about 170 nt, about 171 nt, about 172 nt, about 173 nt, about 174 nt, about 175 nt, about 176 nt, about 177 nt, about 178 nt, about 179 nt, about 180 nt, about 181 nt, about 182 nt, about 183 nt, about 184 nt, about 185 nt, about 186 nt, about 187 nt, about 188 nt, about 189 nt, about 190 nt, about 191 nt, about 192 nt, about 193 nt, about 194 nt, about 195 nt, about 196 nt, about 197 nt, about 198 nt, about 199 nt, about 200 nt, about 201 nt, about 202 nt, about 203 nt, about 204 nt, about 205 nt, about 206 nt, about 207 nt, about 208 nt, about 209 nt, about 210 nt, about 211 nt, about 212 nt, about 213 nt, about 214 nt, about 215 nt, about 216 nt, about 217 nt, about 218 nt, about 219 nt, about 220 nt, about 221 nt, about 222 nt, about 223 nt, about 224 nt, about 225 nt, about 226 nt, about 227 nt, about 228 nt, about 229 nt, about 230 nt, about 231 nt, about 232 nt, about 233 nt, about 234 nt, about 235 nt, about 236 nt, about 237 nt, about 238 nt, about 239 nt, about 240 nt, about 241 nt, about 242 nt, about 243 nt, about 244 nt, about 245 nt, about 246 nt, about 247 nt, about 248 nt, about 249 nt, about 250 nt, about 251 nt, about 252 nt, about 253 nt, about 254 nt, about 255 nt, about 256 nt, about 257 nt, about 258 nt, about 259 nt, about 260 nt, about 261 nt, about 262 nt, about 263 nt, about 264 nt, about 265 nt, about 266 nt, about 267 nt, about 268 nt, about 269 nt, about 270 nt, about 271 nt, about 272 nt, about 273 nt, about 274 nt, about 275 nt, about 276 nt, about 277 nt, about 278 nt, about 279 nt, about 280 nt, about 281 nt, about 282 nt, about 283 nt, about 284 nt, about 285 nt, about 286 nt, about 287 nt, about 288 nt, about 289 nt, about 290 nt, about 291 nt, about 292 nt, about 293 nt, about 294 nt, about 295 nt, about 296 nt, about 297 nt, about 298 nt, about 299 nt, about 300 nt, about 301 nt, about 302 nt, about 303 nt, about 304 nt, about 305 nt, about 306 nt, about 307 nt, about 308 nt, about 309 nt, about 310 nt, about 311 nt, about 312 nt, about 313 nt, about 314 nt, about 315 nt, about 316 nt, about 317 nt, about 318 nt, about 319 nt, about 320 nt, about 321 nt, about 322 nt, about 323 nt, about 324 nt, about 325 nt, about 326 nt, about 327 nt, about 328 nt, about 329 nt, about 330 nt, about 331 nt, about 332 nt, about 333 nt, about 334 nt, about 335 nt, about 336 nt, about 337 nt, about 338 nt, about 339 nt, about 340 nt, about 341 nt, about 342 nt, about 343 nt, about 344 nt, about 345 nt, about 346 nt, about 347 nt, about 348 nt, about 349 nt, about 350 nt, about 351 nt, about 352 nt, about 353 nt, about 354 nt, about 355 nt, about 356 nt, about 357 nt, about 358 nt, about 359 nt, about 360 nt, about 361 nt, about 362 nt, about 363 nt, about 364 nt, about 365 nt, about 366 nt, about 367 nt, about 368 nt, about 369 nt, about 370 nt, about 371 nt, about 372 nt, about 373 nt, about 374 nt, about 375 nt, about 376 nt, about 377 nt, about 378 nt, about 379 nt, about 380 nt, about 381 nt, about 382 nt, about 383 nt, about 384 nt, about 385 nt, about 386 nt, about 387 nt, about 388 nt, about 389 nt, about 390 nt, about 391 nt, about 392 nt, about 393 nt, about 394 nt, about 395 nt, about 396 nt, about 397 nt, about 398 nt, about 399 nt, about 400 nt, about 401 nt, about 402 nt, about 403 nt, about 404 nt, about 405 nt, about 406 nt, about 407 nt, about 408 nt, about 409 nt, about 410 nt, about 411 nt, about 412 nt, about 413 nt, about 414 nt, about 415 nt, about 416 nt, about 417 nt, about 418 nt, about 419 nt, about 420 nt, about 421 nt, about 422 nt, about 423 nt, about 424 nt, about 425 nt, about 426 nt, about 427 nt, about 428 nt, about 429 nt, about 430 nt, about 431 nt, about 432 nt, about 433 nt, about 434 nt, about 435 nt, about 436 nt, about 437 nt, about 438 nt, about 439 nt, about 440 nt, about 441 nt, about 442 nt, about 443 nt, about 444 nt, about 445 nt, about 446 nt, about 447 nt, about 448 nt, about 449 nt, about 450 nt, about 451 nt, about 452 nt, about 453 nt, about 454 nt, about 455 nt, about 456 nt, about 457 nt, about 458 nt, about 459 nt, about 460 nt, about 461 nt, about 462 nt, about 463 nt, about 464 nt, about 465 nt, about 466 nt, about 467 nt, about 468 nt, about 469 nt, about 470 nt, about 471 nt, about 472 nt, about 473 nt, about 474 nt, about 475 nt, about 476 nt, about 477 nt, about 478 nt, about 479 nt, about 480 nt, about 481 nt, about 482 nt, about 483 nt, about 484 nt, about 485 nt, about 486 nt, about 487 nt, about 488 nt, about 489 nt, about 490 nt, about 491 nt, about 492 nt, about 493 nt, about 494 nt, about 495 nt, about 496 nt, about 497 nt, about 498 nt, about 499 nt, or 500 nt.

Some preferred first nucleotide sequences for use in the promoters have a nucleotide sequence that comprises from about 150 nucleotides to about 395 nucleotides from the 3′ end of SEQ ID NO: 1.

The first nucleotide sequence may comprise a nucleic acid sequence having at least 70% sequence identity to SEQ ID NO: 1 over the length of the first nucleotide sequence. The first nucleotide sequence can have a sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to SEQ ID NO: 1 over the length of the first nucleotide sequence. Preferably, the first nucleotide sequence can have at least 90% identity to SEQ ID NO: 1 over the length of the first nucleotide sequence.

The first nucleotide sequence may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, relative to an aligned sequence of the same length from SEQ ID NO: 1.

An exemplary first nucleotide sequence can comprise a sequence of about 395 nt having at least 70% identity to the 3′ end of SEQ ID NO: 1. For example, the first nucleotide sequence can comprise SEQ ID NO: 12. The first nucleotide sequence can comprise a nucleotide sequence that has at least 70% identity to SEQ ID NO:12. An exemplary first nucleotide sequence can comprise a sequence of about 290 nt having at least 70% identity to the 3′ end of SEQ ID NO: 1. For example, the first nucleotide sequence can comprise SEQ ID NO: 13. The first nucleotide sequence can comprise a nucleotide sequence that has at least 70% identity to SEQ ID NO:13. An exemplary first nucleotide sequence can comprise a sequence of about 185 nt having at least 70% identity to the 3′ end of SEQ ID NO: 1. For example, the first nucleotide sequence can comprise SEQ ID NO: 14. The first nucleotide sequence can comprise a nucleotide sequence that has at least 70% identity to SEQ ID NO:14. An exemplary first nucleotide sequence can comprise a sequence of about 150 nt having at least 70% identity to the 3′ end of SEQ ID NO: 1. For example, the first nucleotide sequence can comprise SEQ ID NO: 15. The first nucleotide sequence can comprise a nucleotide sequence that has at least 70% identity to SEQ ID NO:15. In some embodiments, the promoter contains a first nucleotide sequence that comprises SEQ ID NO:12, SEQ ID NO: 13, SEQ ID NO: 14, or SEQ ID NO: 15, and an optional second nucleotide sequence as described herein. In some embodiments, the promoter contains a first nucleotide sequence that comprises SEQ ID NO:12, SEQ ID NO: 13, SEQ ID NO: 14, SEQ ID NO: 15 or a nucleotide sequence that has at least 70% identity to any of the foregoing, and an optional second nucleotide sequence as described herein.

### b. Second Nucleotide Sequence

The promoter can comprise a second nucleotide sequence that is derived from a rod-specific promoter and that as a separate molecule has rod-specific promoter activity. As described herein, when such second nucleotide sequences are included in the hybrid promoters of this disclosure, the resulting hybrid promoter has cone-specific promoter activity. Preferably, the second nucleotide sequence comprises at least about 370 nucleotides and has at least 70% identity to a sequence of equal length from SEQ ID NO: 2. The second nucleotide sequence can comprise at least about 260 nucleotides and has at least 70% identity to a sequence of equal length from SEQ ID NO: 2. A promoter consisting of SEQ ID NO: 2 may herein be referred to as ProA330.

A rod-specific promoter component can be a 5′ truncation, a 3′ truncation, or both of SEQ ID NO: 2 and can contain fewer than the 1000 nt of SEQ ID NO: 2, due to deletion of the nucleotides at the 5′ and/or 3′ ends. For example, the rod-specific promoter component can be the same as nucleotides 106-1000 of SEQ ID NO: 2 or can be the same as nucleotides 1-895 of SEQ ID NO: 2. Preferably, the rod-specific promoter component can comprise or consist of nucleotides 631-895 of SEQ ID NO: 2 (SEQ ID NO:39, which may be referred to as min330), which is believed to be the minimal portion of SEQ ID NO:2 required for rod-specific promoter activity.

A rod-specific promoter component can comprise any number of nucleotides from about 260 to about 369 nucleotides of SEQ ID NO: 2. For example, the rod-specific promoter component can comprise about 261 nt, 262 nt, 263 nt, 264 nt, 265 nt, 266 nt, 267 nt, 268 nt, 269 nt, 270 nt, 271 nt, 272 nt, 272 nt, 274 nt, 275 nt, 276 nt, 277 nt, 278 nt, 279 nt, 280 nt, 281 nt, 282 nt, 283 nt, 284 nt, 285 nt, 286 nt, 287 nt, 288 nt, 289 nt, 290 nt, 291 nt, 292 nt, 293 nt, 294 nt, 295 nt, 296 nt, 297 nt, 298 nt, 299 nt, 300 nt, 301 nt, 302 nt, 303 nt, 304 nt, 305 nt, 306 nt, 307 nt, 308 nt, 309 nt, 310 nt, 311 nt, 312 nt, 313 nt, 314 nt, 315 nt, 316 nt, 317 nt, 318 nt, 319 nt, 320 nt, 321 nt, 322 nt, 323 nt, 324 nt, 325 nt, 326 nt, 327 nt, 328 nt, 329 nt, 330 nt, 331 nt, 332 nt, 333 nt, 334 nt, 335 nt, 336 nt, 337 nt, 338 nt, 339 nt, 340 nt, 341 nt, 342 nt, 343 nt, 344 nt, 345 nt, 346 nt, 347 nt, 348 nt, 349 nt, 350 nt, 351 nt, 352 nt, 353 nt, 354 nt, 355 nt, 356 nt, 357 nt, 358 nt, 359 nt, 360 nt, 361 nt, 362 nt, 363 nt, 364 nt, 365 nt, 366 nt, 367 nt, 368 nt, or about 369 nt of SEQ ID NO: 2. In some preferred aspects, the rod-specific promoter comprises or consist SEQ ID NO: 39 or a sequence that has at least about 70% identity to SEQ ID NO: 39.

For example, a second nucleotide sequence can have a nucleic acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to SEQ ID NO: 2 over the length of the second nucleotide sequence. Preferably, the second nucleotide sequence can have at least 90% identity to SEQ ID NO: 2 over the length of the second nucleotide sequence. These embodiments include, but are not limited to, embodiments wherein the first nucleotide sequence has at least 90% identity to a sequence of equal length from the 3′ end of SEQ ID NO: 1.

Exemplary second nucleotide sequences that can be used in the promoters of this disclosure include SEQ ID NO:2 and a nucleotide sequence that has at least about 90% identity to SEQ ID NO:2 over the length of the second nucleotide sequence.

The second nucleotide sequence may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, relative to an aligned sequence of the same length from SEQ ID NO: 2.

The second nucleotide sequence can comprise a sequence having at least 70% identity to a sequence of equal length from the 3′ end of SEQ ID NO: 2. A second nucleotide sequence according to this paragraph can be considered to be a 5′ truncation of SEQ ID NO: 2, because nucleotides from the 5′ end of SEQ ID NO: 2 have been removed to arrive at the second nucleotide sequence.

The second nucleotide sequence can comprise a sequence having at least 70% identity to a sequence of equal length from the 5′ end of SEQ ID NO: 2. A second nucleotide sequence according to this paragraph can be considered to be a 3′ truncation of SEQ ID NO: 2. because nucleotides from the 3′ end of SEQ ID NO: 2 have been removed to arrive at the second nucleotide sequence.

A second nucleotide sequence can be a 5′ truncation, a 3′ truncation, or both of SEQ ID NO: 2 and can contain fewer than the 1000 nt of SEQ ID NO: 2, due to deletion of nucleotides at the 5′ and/or 3′ ends. For example, the second nucleotide sequence can be the same as nucleotides 106-1000 of SEQ ID NO:2 or can be the same as nucleotides 1-895 of SEQ ID NO:2.

A second nucleotide sequence can comprise any number of nucleotides from 370 to 1000. For example, the second nucleotide sequence can comprise about 370 nt, about 371 nt, about 372 nt, about 373 nt, about 374 nt, about 375 nt, about 376 nt, about 377 nt, about 378 nt, about 379 nt, about 380 nt, about 381 nt, about 382 nt, about 383 nt, about 384 nt, about 385 nt, about 386 nt, about 387 nt, about 388 nt, about 389 nt, about 390 nt, about 391 nt, about 392 nt, about 393 nt, about 394 nt, about 395 nt, about 396 nt, about 397 nt, about 398 nt, about 399 nt, about 400 nt, about 401 nt, about 402 nt, about 403 nt, about 404 nt, about 405 nt, about 406 nt, about 407 nt, about 408 nt, about 409 nt, about 410 nt, about 411 nt, about 412 nt, about 413 nt, about 414 nt, about 415 nt, about 416 nt, about 417 nt, about 418 nt, about 419 nt, about 420 nt, about 421 nt, about 422 nt, about 423 nt, about 424 nt, about 425 nt, about 426 nt, about 427 nt, about 428 nt, about 429 nt, about 430 nt, about 431 nt, about 432 nt, about 433 nt, about 434 nt, about 435 nt, about 436 nt, about 437 nt, about 438 nt, about 439 nt, about 440 nt, about 441 nt, about 442 nt, about 443 nt, about 444 nt, about 445 nt, about 446 nt, about 447 nt, about 448 nt, about 449 nt, about 450 nt, about 451 nt, about 452 nt, about 453 nt, about 454 nt, about 455 nt, about 456 nt, about 457 nt, about 458 nt, about 459 nt, about 460 nt, about 461 nt, about 462 nt, about 463 nt, about 464 nt, about 465 nt, about 466 nt, about 467 nt, about 468 nt, about 469 nt, about 470 nt, about 471 nt, about 472 nt, about 473 nt, about 474 nt, about 475 nt, about 476 nt, about 477 nt, about 478 nt, about 479 nt, about 480 nt, about 481 nt, about 482 nt, about 483 nt, about 484 nt, about 485 nt, about 486 nt, about 487 nt, about 488 nt, about 489 nt, about 490 nt, about 491 nt, about 492 nt, about 493 nt, about 494 nt, about 495 nt, about 496 nt, about 497 nt, about 498 nt, about 499 nt, about 500 nt, about 501 nt, about 502 nt, about 503 nt, about 504 nt, about 505 nt, about 506 nt, about 507 nt, about 508 nt, about 509 nt, about 510 nt, about 511 nt, about 512 nt, about 513 nt, about 514 nt, about 515 nt, about 516 nt, about 517 nt, about 518 nt, about 519 nt, about 520 nt, about 521 nt, about 522 nt, about 523 nt, about 524 nt, about 525 nt, about 526 nt, about 527 nt, about 528 nt, about 529 nt, about 530 nt, about 531 nt, about 532 nt, about 533 nt, about 534 nt, about 535 nt, about 536 nt, about 537 nt, about 538 nt, about 539 nt, about 540 nt, about 541 nt, about 542 nt, about 543 nt, about 544 nt, about 545 nt, about 546 nt, about 547 nt, about 548 nt, about 549 nt, about 550 nt, about 551 nt, about 552 nt, about 553 nt, about 554 nt, about 555 nt, about 556 nt, about 557 nt, about 558 nt, about 559 nt, about 560 nt, about 561 nt, about 562 nt, about 563 nt, about 564 nt, about 565 nt, about 566 nt, about 567 nt, about 568 nt, about 569 nt, about 570 nt, about 571 nt, about 572 nt, about 573 nt, about 574 nt, about 575 nt, about 576 nt, about 577 nt, about 578 nt, about 579 nt, about 580 nt, about 581 nt, about 582 nt, about 583 nt, about 584 nt, about 585 nt, about 586 nt, about 587 nt, about 588 nt, about 589 nt, about 590 nt, about 591 nt, about 592 nt, about 593 nt, about 594 nt, about 595 nt, about 596 nt, about 597 nt, about 598 nt, about 599 nt, about 600 nt, about 601 nt, about 602 nt, about 603 nt, about 604 nt, about 605 nt, about 606 nt, about 607 nt, about 608 nt, about 609 nt, about 610 nt, about 611 nt, about 612 nt, about 613 nt, about 614 nt, about 615 nt, about 616 nt, about 617 nt, about 618 nt, about 619 nt, about 620 nt, about 621 nt, about 622 nt, about 623 nt, about 624 nt, about 625 nt, about 626 nt, about 627 nt, about 628 nt, about 629 nt, about 630 nt, about 631 nt, about 632 nt, about 633 nt, about 634 nt, about 635 nt, about 636 nt, about 637 nt, about 638 nt, about 639 nt, about 640 nt, about 641 nt, about 642 nt, about 643 nt, about 644 nt, about 645 nt, about 646 nt, about 647 nt, about 648 nt, about 649 nt, about 650 nt, about 651 nt, about 652 nt, about 653 nt, about 654 nt, about 655 nt, about 656 nt, about 657 nt, about 658 nt, about 659 nt, about 660 nt, about 661 nt, about 662 nt, about 663 nt, about 664 nt, about 665 nt, about 666 nt, about 667 nt, about 668 nt, about 669 nt, about 670 nt, about 671 nt, about 672 nt, about 673 nt, about 674 nt, about 675 nt, about 676 nt, about 677 nt, about 678 nt, about 679 nt, about 680 nt, about 681 nt, about 682 nt, about 683 nt, about 684 nt, about 685 nt, about 686 nt, about 687 nt, about 688 nt, about 689 nt, about 690 nt, about 691 nt, about 692 nt, about 693 nt, about 694 nt, about 695 nt, about 696 nt, about 697 nt, about 698 nt, about 699 nt, about 700 nt, about 701 nt, about 702 nt, about 703 nt, about 704 nt, about 705 nt, about 706 nt, about 707 nt, about 708 nt, about 709 nt, about 710 nt, about 711 nt, about 712 nt, about 713 nt, about 714 nt, about 715 nt, about 716 nt, about 717 nt, about 718 nt, about 719 nt, about 720 nt, about 721 nt, about 722 nt, about 723 nt, about 724 nt, about 725 nt, about 726 nt, about 727 nt, about 728 nt, about 729 nt, about 730 nt, about 731 nt, about 732 nt, about 733 nt, about 734 nt, about 735 nt, about 736 nt, about 737 nt, about 738 nt, about 739 nt, about 740 nt, about 741 nt, about 742 nt, about 743 nt, about 744 nt, about 745 nt, about 746 nt, about 747 nt, about 748 nt, about 749 nt, about 750 nt, about 751 nt, about 752 nt, about 753 nt, about 754 nt, about 755 nt, about 756 nt, about 757 nt, about 758 nt, about 759 nt, about 760 nt, about 761 nt, about 762 nt, about 763 nt, about 764 nt, about 765 nt, about 766 nt, about 767 nt, about 768 nt, about 769 nt, about 770 nt, about 771 nt, about 772 nt, about 773 nt, about 774 nt, about 775 nt, about 776 nt, about 777 nt, about 778 nt, about 779 nt, about 780 nt, about 781 nt, about 782 nt, about 783 nt, about 784 nt, about 785 nt, about 786 nt, about 787 nt, about 788 nt, about 789 nt, about 790 nt, about 791 nt, about 792 nt, about 793 nt, about 794 nt, about 795 nt, about 796 nt, about 797 nt, about 798 nt, about 799 nt, about 800 nt, about 801 nt, about 802 nt, about 803 nt, about 804 nt, about 805 nt, about 806 nt, about 807 nt, about 808 nt, about 809 nt, about 810 nt, about 811 nt, about 812 nt, about 813 nt, about 814 nt, about 815 nt, about 816 nt, about 817 nt, about 818 nt, about 819 nt, about 820 nt, about 821 nt, about 822 nt, about 823 nt, about 824 nt, about 825 nt, about 826 nt, about 827 nt, about 828 nt, about 829 nt, about 830 nt, about 831 nt, about 832 nt, about 833 nt, about 834 nt, about 835 nt, about 836 nt, about 837 nt, about 838 nt, about 839 nt, about 840 nt, about 841 nt, about 842 nt, about 843 nt, about 844 nt, about 845 nt, about 846 nt, about 847 nt, about 848 nt, about 849 nt, about 850 nt, about 851 nt, about 852 nt, about 853 nt, about 854 nt, about 855 nt, about 856 nt, about 857 nt, about 858 nt, about 859 nt, about 860 nt, about 861 nt, about 862 nt, about 863 nt, about 864 nt, about 865 nt, about 866 nt, about 867 nt, about 868 nt, about 869 nt, about 870 nt, about 871 nt, about 872 nt, about 873 nt, about 874 nt, about 875 nt, about 876 nt, about 877 nt, about 878 nt, about 879 nt, about 880 nt, about 881 nt, about 882 nt, about 883 nt, about 884 nt, about 885 nt, about 886 nt, about 887 nt, about 888 nt, about 889 nt, about 890 nt, about 891 nt, about 892 nt, about 893 nt, about 894 nt, about 895 nt, about 896 nt, about 897 nt, about 898 nt, about 899 nt, about 900 nt, about 901 nt, about 902 nt, about 903 nt, about 904 nt, about 905 nt, about 906 nt, about 907 nt, about 908 nt, about 909 nt, about 910 nt, about 911 nt, about 912 nt, about 913 nt, about 914 nt, about 915 nt, about 916 nt, about 917 nt, about 918 nt, about 919 nt, about 920 nt, about 921 nt, about 922 nt, about 923 nt, about 924 nt, about 925 nt, about 926 nt, about 927 nt, about 928 nt, about 929 nt, about 930 nt, about 931 nt, about 932 nt, about 933 nt, about 934 nt, about 935 nt, about 936 nt, about 937 nt, about 938 nt, about 939 nt, about 940 nt, about 941 nt, about 942 nt, about 943 nt, about 944 nt, about 945 nt, about 946 nt, about 947 nt, about 948 nt, about 949 nt, about 950 nt, about 951 nt, about 952 nt, about 953 nt, about 954 nt, about 955 nt, about 956 nt, about 957 nt, about 958 nt, about 959 nt, about 960 nt, about 961 nt, about 962 nt, about 963 nt, about 964 nt, about 965 nt, about 966 nt, about 967 nt, about 968 nt, about 969 nt, about 970 nt, about 971 nt, about 972 nt, about 973 nt, about 974 nt, about 975 nt, about 976 nt, about 977 nt, about 978 nt, about 979 nt, about 980 nt, about 981 nt, about 982 nt, about 983 nt, about 984 nt, about 985 nt, about 986 nt, about 987 nt, about 988 nt, about 989 nt, about 990 nt, about 991 nt, about 992 nt, about 993 nt, about 994 nt, about 995 nt, about 996 nt, about 997 nt, about 998 nt, about 999 nt, or about 1000 nt.

Some preferred second nucleotide sequences for use in the promoters of this disclosure have a nucleotide sequence that comprises from about 370 nucleotides to about 1000 nucleotides from the 3′ end of SEQ ID NO: 2. Some preferred second nucleotide sequences for use in the promoters of this disclosure have a nucleotide sequence that comprises from about 895 nucleotides to about 1000 nucleotides from the 5′ end of SEQ ID NO: 2. Particularly preferred second nucleotides comprise SEQ ID NO: 39 or a sequence that has at least about 70% identity to SEQ ID NO: 39.

The second nucleotide sequence may comprise a nucleic acid sequence having at least 70% sequence identity to SEQ ID NO: 2 over the length of the second nucleotide sequence. The second nucleotide sequence can have a sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to SEQ ID NO: 2 over the length of the second nucleotide sequence. Preferably, the second nucleotide sequence can have at least 90% identity to SEQ ID NO: 2 over the length of the second nucleotide sequence.

The second nucleotide sequence may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, relative to an aligned sequence of the same length from SEQ ID NO: 2.

An exemplary second nucleotide sequence can comprise a sequence of about 895 nt having at least 70% identity to the 3′ end of SEQ ID NO: 2. For example, the second nucleotide sequence can comprise SEQ ID NO: 21. An exemplary second nucleotide sequence can comprise a sequence of about 790 nt having at least 70% identity to the 3′ end of SEQ ID NO: 2. For example, the second nucleotide sequence can comprise SEQ ID NO: 22. An exemplary second nucleotide sequence can comprise a sequence of about 685 nt having at least 70% identity to the 3′ end of SEQ ID NO: 2. For example, the second nucleotide sequence can comprise SEQ ID NO: 23. An exemplary second nucleotide sequence can comprise a sequence of about 580 nt having at least 70% identity to the 3′ end of SEQ ID NO: 2. For example, the second nucleotide sequence can comprise SEQ ID NO: 24. An exemplary second nucleotide sequence can comprise a sequence of about 475 nt having at least 70% identity to the 3′ end of SEQ ID NO: 2. For example, the second nucleotide sequence can comprise SEQ ID NO: 25. An exemplary second nucleotide sequence can comprise a sequence of about 390 nt having at least 70% identity to the 3′ end of SEQ ID NO: 2. For example, the second nucleotide sequence can comprise SEQ ID NO: 26. An exemplary second nucleotide sequence can comprise a sequence of about 895 nt having at least 70% identity to the 5′ end of SEQ ID NO: 2. For example, the second nucleotide sequence can comprise SEQ ID NO: 30.

In some embodiments, the second nucleotide sequence comprises SEQ ID NO: 21, SEQ ID NO: 22, SEQ ID NO: 23, SEQ ID NO: 24, SEQ ID NO: 25, SEQ ID NO: 26, or SEQ ID NO: 30. In some embodiments, the second nucleotide sequence comprises a sequence that has at least 70% identity to SEQ ID NO: 21, SEQ ID NO: 22, SEQ ID NO: 23, SEQ ID NO: 24, SEQ ID NO: 25, SEQ ID NO: 26, or SEQ ID NO: 30. In preferred embodiments, the second nucleotide sequence comprises SEQ ID NO: 39 or a sequence that has at least about 70% identity to SEQ ID NO: 39.

### c. Hybrid Promoter Structure and Orientation

As described and exemplified herein, a hybrid promoter comprising a first nucleotide sequence which is derived from a cone-specific promoter (SEQ ID NO: 1) and a second nucleotide sequence which is derived from a rod-specific promoter (SEQ ID NO: 2) has two unexpected properties: it is cone-specific and has increased promoter activity compared to full length SEQ ID NO: 1 alone.

The hybrid promoter comprises a first nucleotide sequence and a second nucleotide sequence, both as described herein. If desired, the promoter can further comprise one or more other rod-specific or cone-specific promoter sequences. In some embodiments, the promoter can further comprise one or more other cone-specific promoters or nucleotide sequences derived from a cone-specific promoter, such as hG1.7 (SEQ ID NO: 20) or PR1.7 (SEQ ID NO: 19).

The first nucleotide sequence, the second nucleotide sequence, and any other rod- or cone-specific promoter sequences (if included), can each be included in the promoter as a single copy or as multiple copies. For example, each first nucleotide sequence and each second nucleotide sequence can be present in one to about 10 copies (e.g., 1, 2, 3, 4, 5, 6, 7, 8, 9 or 10 copies). In some embodiments, the promoter can comprise two or more first nucleotide sequences, two or more second nucleotide sequences, or both. When two or more first nucleotide sequences are included in the promoter, the two first nucleotide sequences need not be identical, provided they each individually meet the criteria set forth herein regarding first nucleotide sequences. Similarly, when two or more second nucleotide sequences are included in the promoter, the two second nucleotide sequences need not be identical, provided they each individually meet the criteria set forth herein regarding second nucleotide sequences.

In the hybrid promoters of this disclosure, the first nucleotide sequence(s) and the second nucleotide sequence(s) can be positioned in any desired order, and with or without other sequences therebetween. As exemplified herein, hybrid promoters that include the same first nucleotide sequence and the same second nucleotide sequence, but in different orders, are effective at driving expression of reporter genes in human cone photoreceptors.

The promoter can comprise from 5′ to 3′ the first nucleotide sequence linked to the second nucleotide sequence with no sequences therebetween. The promoter can comprise from 5′ to 3′ the second nucleotide sequence linked to the first nucleotide sequence with no sequences therebetween. In other examples, the promoter can comprise two first nucleotide sequences (first sequence A and first sequence B) and one second nucleotide sequence. arranged first sequence A-second nucleotide sequence-first sequence B from 5′ to 3′.

In some embodiments, the promoter can comprise one or more promoter units, wherein each promoter unit comprises a first nucleotide sequence and a second nucleotide sequence in either order, and each promoter unit as a separate molecule has cone-specific promoter activity. For example, the promoter can comprise from 1 to about 4 promoter units. In another example, the promoter can comprise from 4 to 10 promoter units. The promoter can comprise 1, 2, 3, 4, 5, 6, 7, 8, 9 or 10 promoter units that can be the same or different.

Some particular promoters can include, from 5′ to 3′, a first nucleotide sequence comprising SEQ ID NO: 1 and a second nucleotide sequence comprising SEQ ID NO: 2. A particular such promoter, termed Pro572, has SEQ ID NO: 3. In some embodiments, the first nucleotide sequence comprises a sequence that has at least 70% identity to SEQ ID NO: 3. Some particular promoters can include, from 5′ to 3′, a second nucleotide sequence comprising SEQ ID NO: 2 and a first nucleotide sequence comprising SEQ ID NO: 1. A particular such promoter, termed Pro573, has SEQ ID NO: 5. In some embodiments, the first nucleotide sequence comprises a sequence that has at least 70% identity to SEQ ID NO: 5. Some particular promoters can include, from 5′ to 3′, a first nucleotide sequence comprising SEQ ID NO: 12 and a second nucleotide sequence comprising SEQ ID NO: 2. Some particular promoters can include, from 5′ to 3′, a second nucleotide sequence comprising SEQ ID NO: 2 and a first nucleotide sequence comprising SEQ ID NO: 12. Some particular promoters can include, from 5′ to 3′, a first nucleotide sequence comprising SEQ ID NO: 13 and a second nucleotide sequence comprising SEQ ID NO: 2. Some particular promoters can include, from 5′ to 3′, a second nucleotide sequence comprising SEQ ID NO: 2 and a first nucleotide sequence comprising SEQ ID NO: 13. Some particular promoters can include, from 5′ to 3′, a first nucleotide sequence comprising SEQ ID NO: 14 and a second nucleotide sequence comprising SEQ ID NO: 2. A particular such promoter, termed Pro572.2, has SEQ ID NO: 4. In some embodiments, the nucleotide sequence for a promoter comprises a sequence that has at least 70% identity to SEQ ID NO: 4.

In some embodiments, a promoter can include, from 5′ to 3′, a second nucleotide sequence comprising SEQ ID NO: 2 and a first nucleotide sequence comprising SEQ ID NO: 14. A particular such promoter, termed Pro573.2, has SEQ ID NO: 6. In some embodiments, the nucleotide sequence for a promoter comprises a sequence that has at least 70% identity to SEQ ID NO: 6. Some particular promoters can include, from 5′ to 3′, a first nucleotide sequence comprising SEQ ID NO: 15 and a second nucleotide sequence comprising SEQ ID NO: 2. Some particular promoters can include, from 5′ to 3′, a second nucleotide sequence comprising SEQ ID NO: 2 and a first nucleotide sequence comprising SEQ ID NO: 15. Some particular promoters can include, from 5′ to 3′, a first nucleotide sequence comprising SEQ ID NO: 1 and a second nucleotide sequence comprising SEQ ID NO: 21. Some particular promoters can include, from 5′ to 3′, a second nucleotide sequence comprising SEQ ID NO: 21 and a first nucleotide sequence comprising SEQ ID NO: 1. Some particular promoters can include, from 5′ to 3′, a first nucleotide sequence comprising SEQ ID NO: 1 and a second nucleotide sequence comprising SEQ ID NO: 22. Some particular promoters can include, from 5′ to 3′, a second nucleotide sequence comprising SEQ ID NO: 22 and a first nucleotide sequence comprising SEQ ID NO: 1. Some particular promoters can include, from 5′ to 3′, a first nucleotide sequence comprising SEQ ID NO: 1 and a second nucleotide sequence comprising SEQ ID NO: 23. Some particular promoters can include, from 5′ to 3′, a second nucleotide sequence comprising SEQ ID NO: 23 and a first nucleotide sequence comprising SEQ ID NO: 1. Some particular promoters can include, from 5′ to 3′, a first nucleotide sequence comprising SEQ ID NO: 1 and a second nucleotide sequence comprising SEQ ID NO: 24. Some particular promoters can include, from 5′ to 3′, a second nucleotide sequence comprising SEQ ID NO: 24 and a first nucleotide sequence comprising SEQ ID NO: 1. Some particular promoters can include, from 5′ to 3′, a first nucleotide sequence comprising SEQ ID NO: 1 and a second nucleotide sequence comprising SEQ ID NO: 25. Some particular promoters can include, from 5′ to 3′, a second nucleotide sequence comprising SEQ ID NO: 25 and a first nucleotide sequence comprising SEQ ID NO: 1. Some particular promoters can include, from 5′ to 3′, a first nucleotide sequence comprising SEQ ID NO: 1 and a second nucleotide sequence comprising SEQ ID NO: 26. Some particular promoters can include, from 5′ to 3′, a second nucleotide sequence comprising SEQ ID NO: 26 and a first nucleotide sequence comprising SEQ ID NO: 1. Some particular promoters can include, from 5′ to 3′, a first nucleotide sequence comprising SEQ ID NO: 1 and a second nucleotide sequence comprising SEQ ID NO: 30. Some particular promoters can include, from 5′ to 3′, a second nucleotide sequence comprising SEQ ID NO: 30 and a first nucleotide sequence comprising SEQ ID NO: 1. Some particular promoters can include, from 5′ to 3′, a first nucleotide sequence comprising SEQ ID NO: 12 and a second nucleotide sequence comprising SEQ ID NO: 21. Some particular promoters can include, from 5′ to 3′, a second nucleotide sequence comprising SEQ ID NO: 21 and a first nucleotide sequence comprising SEQ ID NO: 12. Some particular promoters can include, from 5′ to 3′, a first nucleotide sequence comprising SEQ ID NO: 12 and a second nucleotide sequence comprising SEQ ID NO: 22. Some particular promoters can include, from 5′ to 3′, a second nucleotide sequence comprising SEQ ID NO: 22 and a first nucleotide sequence comprising SEQ ID NO: 12. Some particular promoters can include, from 5′ to 3′, a first nucleotide sequence comprising SEQ ID NO: 12 and a second nucleotide sequence comprising SEQ ID NO: 23. Some particular promoters can include, from 5′ to 3′, a second nucleotide sequence comprising SEQ ID NO: 23 and a first nucleotide sequence comprising SEQ ID NO: 12. Some particular promoters can include, from 5′ to 3′, a first nucleotide sequence comprising SEQ ID NO: 12 and a second nucleotide sequence comprising SEQ ID NO: 24. Some particular promoters can include, from 5′ to 3′, a second nucleotide sequence comprising SEQ ID NO: 24 and a first nucleotide sequence comprising SEQ ID NO: 12. Some particular promoters can include, from 5′ to 3′, a first nucleotide sequence comprising SEQ ID NO: 12 and a second nucleotide sequence comprising SEQ ID NO: 25. Some particular promoters can include, from 5′ to 3′, a second nucleotide sequence comprising SEQ ID NO: 25 and a first nucleotide sequence comprising SEQ ID NO: 12. Some particular promoters can include, from 5′ to 3′, a first nucleotide sequence comprising SEQ ID NO: 12 and a second nucleotide sequence comprising SEQ ID NO: 26. Some particular promoters can include, from 5′ to 3′, a second nucleotide sequence comprising SEQ ID NO: 26 and a first nucleotide sequence comprising SEQ ID NO: 12. Some particular promoters can include, from 5′ to 3′, a first nucleotide sequence comprising SEQ ID NO: 12 and a second nucleotide sequence comprising SEQ ID NO: 30. Some particular promoters can include, from 5′ to 3′, a second nucleotide sequence comprising SEQ ID NO: 30 and a first nucleotide sequence comprising SEQ ID NO: 12. Some particular promoters can include, from 5′ to 3′, a first nucleotide sequence comprising SEQ ID NO: 13 and a second nucleotide sequence comprising SEQ ID NO: 21. Some particular promoters can include, from 5′ to 3′, a second nucleotide sequence comprising SEQ ID NO: 21 and a first nucleotide sequence comprising SEQ ID NO: 13. Some particular promoters can include, from 5′ to 3′, a first nucleotide sequence comprising SEQ ID NO: 13 and a second nucleotide sequence comprising SEQ ID NO: 22. Some particular promoters can include, from 5′ to 3′, a second nucleotide sequence comprising SEQ ID NO: 22 and a first nucleotide sequence comprising SEQ ID NO: 13. Some particular promoters can include, from 5′ to 3′, a first nucleotide sequence comprising SEQ ID NO: 13 and a second nucleotide sequence comprising SEQ ID NO: 23. Some particular promoters can include, from 5′ to 3′, a second nucleotide sequence comprising SEQ ID NO: 23 and a first nucleotide sequence comprising SEQ ID NO: 13. Some particular promoters can include, from 5′ to 3′, a first nucleotide sequence comprising SEQ ID NO: 13 and a second nucleotide sequence comprising SEQ ID NO: 24. Some particular promoters can include, from 5′ to 3′, a second nucleotide sequence comprising SEQ ID NO: 24 and a first nucleotide sequence comprising SEQ ID NO: 13. Some particular promoters can include, from 5′ to 3′, a first nucleotide sequence comprising SEQ ID NO: 13 and a second nucleotide sequence comprising SEQ ID NO: 25. Some particular promoters can include, from 5′ to 3′, a second nucleotide sequence comprising SEQ ID NO: 25 and a first nucleotide sequence comprising SEQ ID NO: 13. Some particular promoters can include, from 5′ to 3′, a first nucleotide sequence comprising SEQ ID NO: 13 and a second nucleotide sequence comprising SEQ ID NO: 26. Some particular promoters can include, from 5′ to 3′, a second nucleotide sequence comprising SEQ ID NO: 26 and a first nucleotide sequence comprising SEQ ID NO: 13. Some particular promoters can include, from 5′ to 3′, a first nucleotide sequence comprising SEQ ID NO: 13 and a second nucleotide sequence comprising SEQ ID NO: 30. Some particular promoters can include, from 5′ to 3′, a second nucleotide sequence comprising SEQ ID NO: 30 and a first nucleotide sequence comprising SEQ ID NO: 13. Some particular promoters can include, from 5′ to 3′, a first nucleotide sequence comprising SEQ ID NO: 14 and a second nucleotide sequence comprising SEQ ID NO: 21. Some particular promoters can include, from 5′ to 3′, a second nucleotide sequence comprising SEQ ID NO: 21 and a first nucleotide sequence comprising SEQ ID NO: 14. Some particular promoters can include, from 5′ to 3′, a first nucleotide sequence comprising SEQ ID NO: 14 and a second nucleotide sequence comprising SEQ ID NO: 22. Some particular promoters can include, from 5′ to 3′, a second nucleotide sequence comprising SEQ ID NO: 22 and a first nucleotide sequence comprising SEQ ID NO: 14. Some particular promoters can include, from 5′ to 3′, a first nucleotide sequence comprising SEQ ID NO: 14 and a second nucleotide sequence comprising SEQ ID NO: 23. Some particular promoters can include, from 5′ to 3′, a second nucleotide sequence comprising SEQ ID NO: 23 and a first nucleotide sequence comprising SEQ ID NO: 14. Some particular promoters can include, from 5′ to 3′, a first nucleotide sequence comprising SEQ ID NO: 14 and a second nucleotide sequence comprising SEQ ID NO: 24. Some particular promoters can include, from 5′ to 3′, a second nucleotide sequence comprising SEQ ID NO: 24 and a first nucleotide sequence comprising SEQ ID NO: 14. Some particular promoters can include, from 5′ to 3′, a first nucleotide sequence comprising SEQ ID NO: 14 and a second nucleotide sequence comprising SEQ ID NO: 25. Some particular promoters can include, from 5′ to 3′, a second nucleotide sequence comprising SEQ ID NO: 25 and a first nucleotide sequence comprising SEQ ID NO: 14. Some particular promoters can include, from 5′ to 3′, a first nucleotide sequence comprising SEQ ID NO: 14 and a second nucleotide sequence comprising SEQ ID NO: 26. Some particular promoters can include, from 5′ to 3′, a second nucleotide sequence comprising SEQ ID NO: 26 and a first nucleotide sequence comprising SEQ ID NO: 14. Some particular promoters can include, from 5′ to 3′, a first nucleotide sequence comprising SEQ ID NO: 14 and a second nucleotide sequence comprising SEQ ID NO: 30. Some particular promoters can include, from 5′ to 3′, a second nucleotide sequence comprising SEQ ID NO: 30 and a first nucleotide sequence comprising SEQ ID NO: 14. Some particular promoters can include, from 5′ to 3′, a first nucleotide sequence comprising SEQ ID NO: 15 and a second nucleotide sequence comprising SEQ ID NO: 21. Some particular promoters can include, from 5′ to 3′, a second nucleotide sequence comprising SEQ ID NO: 21 and a first nucleotide sequence comprising SEQ ID NO: 15. Some particular promoters can include, from 5′ to 3′, a first nucleotide sequence comprising SEQ ID NO: 15 and a second nucleotide sequence comprising SEQ ID NO: 22. Some particular promoters can include, from 5′ to 3′, a second nucleotide sequence comprising SEQ ID NO: 22 and a first nucleotide sequence comprising SEQ ID NO: 15. Some particular promoters can include, from 5′ to 3′, a first nucleotide sequence comprising SEQ ID NO: 15 and a second nucleotide sequence comprising SEQ ID NO: 23. Some particular promoters can include, from 5′ to 3′, a second nucleotide sequence comprising SEQ ID NO: 23 and a first nucleotide sequence comprising SEQ ID NO: 15. Some particular promoters can include, from 5′ to 3′, a first nucleotide sequence comprising SEQ ID NO: 15 and a second nucleotide sequence comprising SEQ ID NO: 24. Some particular promoters can include, from 5′ to 3′, a second nucleotide sequence comprising SEQ ID NO: 24 and a first nucleotide sequence comprising SEQ ID NO: 15. Some particular promoters can include, from 5′ to 3′, a first nucleotide sequence comprising SEQ ID NO: 15 and a second nucleotide sequence comprising SEQ ID NO: 25. Some particular promoters can include, from 5′ to 3′, a second nucleotide sequence comprising SEQ ID NO: 25 and a first nucleotide sequence comprising SEQ ID NO: 15. Some particular promoters can include, from 5′ to 3′, a first nucleotide sequence comprising SEQ ID NO: 15 and a second nucleotide sequence comprising SEQ ID NO: 26. Some particular promoters can include, from 5′ to 3′, a second nucleotide sequence comprising SEQ ID NO: 26 and a first nucleotide sequence comprising SEQ ID NO: 15. Some particular promoters can include, from 5′ to 3′, a first nucleotide sequence comprising SEQ ID NO: 15 and a second nucleotide sequence comprising SEQ ID NO: 30. Some particular promoters can include, from 5′ to 3′, a second nucleotide sequence comprising SEQ ID NO: 30 and a first nucleotide sequence comprising SEQ ID NO: 15. In certain preferred embodiments, the hybrid promoter contains about 700 or fewer nucleotides, about 600 or fewer nucleotides, or more preferably, about 500 or fewer nucleotides.

### d. Cone-Specific Promoters that are Truncations of SEQ ID NO: 1

In other aspects, the cone-specific synthetic promoters disclosed herein comprise an active fragment of the cone-specific promoter of SEQ ID NO: 1, with the proviso that the synthetic promoter of this aspect is not SEQ ID NO:1. The nucleic acids disclosed herein can comprise a promoter comprising a cone-specific promoter of at least about 150 nucleotides and no more than 499 nucleotides which has at least 70% identity to SEQ ID NO: 1 over the length of the cone-specific promoter. Such promoters can lack a second nucleotide sequence, as described herein.

As disclosed and exemplified herein, truncations of SEQ ID NO: 1 have cone-specific promoter activity and can drive expression in human cone cells, for example, cone cells that are dormant and no longer responsive to light. Moreover, such promoters can drive higher expression in cone cells than the full-length SEQ ID NO: 1 alone. Such promoters can have promoter activity in human cone cells, for example cone cells of the retina in vivo, in retinal explants or retinal organoids. In some instances, the cone-specific promoter does not have promoter activity in other cells of the human retina, such as rod cells.

The cone-specific promoter may comprise a nucleic acid sequence having at least 70% sequence identity to a sequence of equal length (i.e., at least about 150 nt and no more than 499 nt) from SEQ ID NO: 1. The first sequence can have a nucleic acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or greater sequence identity to SEQ ID NO: 1 over the length of the first nucleotide sequence.

The cone-specific promoter may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, compared to the corresponding portion of SEQ ID NO:1.

The cone-specific promoter can comprise at least 150 nucleotides, which have at least 70% identity to SEQ ID NO: 1, preferably the 3′ end of SEQ ID NO: 1. As shown herein, such fragments of SEQ ID NO:1 retain cone-specific promoter activity.

The cone-specific promoter can comprise at least 150 nucleotides, which have at least 70% identity to a sequence of equal length from the 3′ end of SEQ ID NO: 1. A cone-specific promoter according to this paragraph can be considered to be a 5′ truncation of SEQ ID NO: 1, because nucleotides from the 5′ end of SEQ ID NO: 1 have been removed to arrive at the first sequence.

The cone-specific promoter can be a 5′ truncation of SEQ ID NO: 1 and contain at least 150 nt but fewer than the 500 nt of ProA7, due to deletion of nucleotides at the 5′ end. For example, the cone-specific promoter can be nucleotides 386-500 of SEQ ID NO:1 or nucleotides 2-500 of SEQ ID NO:1.

For example, the cone-specific promoter can comprise about 150 nucleotides (nt), about 151 nt, about 152 nt, about 153 nt, about 154 nt, about 155 nt, about 156 nt, about 157 nt, about 158 nt, about 159 nt, about 160 nt, about 161 nt, about 162 nt, about 163 nt, about 164 nt, about 165 nt, about 166 nt, about 167 nt, about 168 nt, about 169 nt, about 170 nt, about 171 nt, about 172 nt, about 173 nt, about 174 nt, about 175 nt, about 176 nt, about 177 nt, about 178 nt, about 179 nt, about 180 nt, about 181 nt, about 182 nt, about 183 nt, about 184 nt, about 185 nt, about 186 nt, about 187 nt, about 188 nt, about 189 nt, about 190 nt, about 191 nt, about 192 nt, about 193 nt, about 194 nt, about 195 nt, about 196 nt, about 197 nt, about 198 nt, about 199 nt, about 200 nt, about 201 nt, about 202 nt, about 203 nt, about 204 nt, about 205 nt, about 206 nt, about 207 nt, about 208 nt, about 209 nt, about 210 nt, about 211 nt, about 212 nt, about 213 nt, about 214 nt, about 215 nt, about 216 nt, about 217 nt, about 218 nt, about 219 nt, about 220 nt, about 221 nt, about 222 nt, about 223 nt, about 224 nt, about 225 nt, about 226 nt, about 227 nt, about 228 nt, about 229 nt, about 230 nt, about 231 nt, about 232 nt, about 233 nt, about 234 nt, about 235 nt, about 236 nt, about 237 nt, about 238 nt, about 239 nt, about 240 nt, about 241 nt, about 242 nt, about 243 nt, about 244 nt, about 245 nt, about 246 nt, about 247 nt, about 248 nt, about 249 nt, about 250 nt, about 251 nt, about 252 nt, about 253 nt, about 254 nt, about 255 nt, about 256 nt, about 257 nt, about 258 nt, about 259 nt, about 260 nt, about 261 nt, about 262 nt, about 263 nt, about 264 nt, about 265 nt, about 266 nt, about 267 nt, about 268 nt, about 269 nt, about 270 nt, about 271 nt, about 272 nt, about 273 nt, about 274 nt, about 275 nt, about 276 nt, about 277 nt, about 278 nt, about 279 nt, about 280 nt, about 281 nt, about 282 nt, about 283 nt, about 284 nt, about 285 nt, about 286 nt, about 287 nt, about 288 nt, about 289 nt, about 290 nt, about 291 nt, about 292 nt, about 293 nt, about 294 nt, about 295 nt, about 296 nt, about 297 nt, about 298 nt, about 299 nt, about 300 nt, about 301 nt, about 302 nt, about 303 nt, about 304 nt, about 305 nt, about 306 nt, about 307 nt, about 308 nt, about 309 nt, about 310 nt, about 311 nt, about 312 nt, about 313 nt, about 314 nt, about 315 nt, about 316 nt, about 317 nt, about 318 nt, about 319 nt, about 320 nt, about 321 nt, about 322 nt, about 323 nt, about 324 nt, about 325 nt, about 326 nt, about 327 nt, about 328 nt, about 329 nt, about 330 nt, about 331 nt, about 332 nt, about 333 nt, about 334 nt, about 335 nt, about 336 nt, about 337 nt, about 338 nt, about 339 nt, about 340 nt, about 341 nt, about 342 nt, about 343 nt, about 344 nt, about 345 nt, about 346 nt, about 347 nt, about 348 nt, about 349 nt, about 350 nt, about 351 nt, about 352 nt, about 353 nt, about 354 nt, about 355 nt, about 356 nt, about 357 nt, about 358 nt, about 359 nt, about 360 nt, about 361 nt, about 362 nt, about 363 nt, about 364 nt, about 365 nt, about 366 nt, about 367 nt, about 368 nt, about 369 nt, about 370 nt, about 371 nt, about 372 nt, about 373 nt, about 374 nt, about 375 nt, about 376 nt, about 377 nt, about 378 nt, about 379 nt, about 380 nt, about 381 nt, about 382 nt, about 383 nt, about 384 nt, about 385 nt, about 386 nt, about 387 nt, about 388 nt, about 389 nt, about 390 nt, about 391 nt, about 392 nt, about 393 nt, about 394 nt, about 395 nt, about 396 nt, about 397 nt, about 398 nt, about 399 nt, about 400 nt, about 401 nt, about 402 nt, about 403 nt, about 404 nt, about 405 nt, about 406 nt, about 407 nt, about 408 nt, about 409 nt, about 410 nt, about 411 nt, about 412 nt, about 413 nt, about 414 nt, about 415 nt, about 416 nt, about 417 nt, about 418 nt, about 419 nt, about 420 nt, about 421 nt, about 422 nt, about 423 nt, about 424 nt, about 425 nt, about 426 nt, about 427 nt, about 428 nt, about 429 nt, about 430 nt, about 431 nt, about 432 nt, about 433 nt, about 434 nt, about 435 nt, about 436 nt, about 437 nt, about 438 nt, about 439 nt, about 440 nt, about 441 nt, about 442 nt, about 443 nt, about 444 nt, about 445 nt, about 446 nt, about 447 nt, about 448 nt, about 449 nt, about 450 nt, about 451 nt, about 452 nt, about 453 nt, about 454 nt, about 455 nt, about 456 nt, about 457 nt, about 458 nt, about 459 nt, about 460 nt, about 461 nt, about 462 nt, about 463 nt, about 464 nt, about 465 nt, about 466 nt, about 467 nt, about 468 nt, about 469 nt, about 470 nt, about 471 nt, about 472 nt, about 473 nt, about 474 nt, about 475 nt, about 476 nt, about 477 nt, about 478 nt, about 479 nt, about 480 nt, about 481 nt, about 482 nt, about 483 nt, about 484 nt, about 485 nt, about 486 nt, about 487 nt, about 488 nt, about 489 nt, about 490 nt, about 491 nt, about 492 nt, about 493 nt, about 494 nt, about 495 nt, about 496 nt, about 497 nt, about 498 nt, or about 499 nt of SEQ ID NO:1.

Some preferred cone-specific promoters of this aspect of the disclosure have a nucleotide sequence that comprises from about 150 nucleotides to about 395 nucleotides from the 3′ end of SEQ ID NO: 1.

As stated, the cone-specific promoter may comprise a nucleic acid sequence having at least 70% sequence identity to SEQ ID NO: 1 over the length of the cone-specific promoter. The cone-specific promoter can have a sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to SEQ ID NO: 1 over the length of the cone-specific promoter. Preferably, the cone-specific promoter can have at least 90% identity to SEQ ID NO: 1 over the length of the cone-specific promoter.

The cone-specific promoter may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, relative to an aligned sequence of the same length from SEQ ID NO: 1.

An exemplary cone-specific promoter can comprise a sequence of about 395 nt having at least 70% identity to the 3′ end of SEQ ID NO: 1. For example, the cone-specific promoter can comprise SEQ ID NO: 12. The cone-specific promoter can comprise a nucleotide sequence that has at least 70% identity to SEQ ID NO: 12. An exemplary cone-specific promoter can comprise a sequence of about 290 nt having at least 70% identity to the 3′ end of SEQ ID NO: 1. For example, the cone-specific promoter can comprise SEQ ID NO: 13. The cone-specific promoter can comprise a nucleotide sequence that has at least 70% identity to SEQ ID NO: 13. An exemplary cone-specific promoter can comprise a sequence of about 185 nt having at least 70% identity to the 3′ end of SEQ ID NO: 1. For example, the cone-specific promoter can comprise SEQ ID NO: 14 which can be referred to as ProSC. The cone-specific promoter can comprise a nucleotide sequence that has at least 70% identity to SEQ ID NO: 14. An exemplary cone-specific promoter can comprise a sequence of about 150 nt having at least 70% identity to the 3′ end of SEQ ID NO: 1. For example, the cone-specific promoter can comprise SEQ ID NO: 15. The cone-specific promoter can comprise a nucleotide sequence that has at least 70% identity to SEQ ID NO: 15.

In some embodiments, the promoter contains a cone-specific promoter that comprises SEQ ID NO:12, SEQ ID NO: 13, SEQ ID NO: 14, or SEQ ID NO: 15.

The cone-specific promoters can be present in one or more copies. Alternatively or in addition, sequences from one or more other cone-specific promoters can be included. Examples of such other promotes include hG1.7 (SEQ ID NO: 20) and PR1.7 (SEQ ID NO: 19).

The cone-specific promoter and any other cone-specific promoter sequences (if included), can each be included in the promoter as a single copy or as multiple copies. In some embodiments, the promoter can comprise two or more cone-specific promoters. When two or more cone-specific promoters are included in the promoter, the two cone-specific promoters need not be identical, provided they each individually meet the criteria set forth herein regarding cone-specific promoters. For example, each cone-specific promoter can be present in one to about 10 copies (e.g., 1, 2, 3, 4, 5, 6, 7, 8, 9 or 10 copies).

In promoters lacking second nucleotide sequences and comprising two or more cone-specific promoters, the cone-specific promoter(s) can be positioned in any desired order, and with or without any other sequences therebetween.

In some embodiments, the promoter can comprise one or more units, wherein each unit comprises one or more cone-specific promoter in any desired order. For example, the promoter can comprise from 1 to about 4 promoter units of cone-specific promoter(s).

In some embodiments, the promoter can comprise about 1, about 2, about 3, or about 4 units each comprising, from 5′ to 3′, SEQ ID NO: 12 and SEQ ID NO: 12. In some embodiments, the promoter can comprise about 1, about 2, about 3, or about 4 units each comprising, from 5′ to 3′, SEQ ID NO: 12 and SEQ ID NO: 13. In some embodiments, the promoter can comprise about 1, about 2, about 3, or about 4 units each comprising, from 5′ to 3′, SEQ ID NO: 13 and SEQ ID NO: 12. In some embodiments, the promoter can comprise about 1, about 2, about 3, or about 4 units each comprising, from 5′ to 3′, SEQ ID NO: 12 and SEQ ID NO: 14. In some embodiments, the promoter can comprise about 1, about 2, about 3, or about 4 units each comprising, from 5′ to 3′, SEQ ID NO: 14 and SEQ ID NO: 12. In some embodiments, the promoter can comprise about 1, about 2, about 3, or about 4 units each comprising, from 5′ to 3′, SEQ ID NO: 12 and SEQ ID NO: 15. In some embodiments, the promoter can comprise about 1, about 2, about 3, or about 4 units each comprising, from 5′ to 3′, SEQ ID NO: 15 and SEQ ID NO: 12. In some embodiments, the promoter can comprise about 1, about 2, about 3, or about 4 units each comprising, from 5′ to 3′, SEQ ID NO: 13 and SEQ ID NO: 12. In some embodiments, the promoter can comprise about 1, about 2, about 3, or about 4 units each comprising, from 5′ to 3′, SEQ ID NO: 12 and SEQ ID NO: 13. In some embodiments, the promoter can comprise about 1, about 2, about 3, or about 4 units each comprising, from 5′ to 3′, SEQ ID NO: 13 and SEQ ID NO: 13. In some embodiments, the promoter can comprise about 1, about 2, about 3, or about 4 units each comprising, from 5′ to 3′, SEQ ID NO: 13 and SEQ ID NO: 14. In some embodiments, the promoter can comprise about 1, about 2, about 3, or about 4 units each comprising, from 5′ to 3′, SEQ ID NO: 14 and SEQ ID NO: 13. In some embodiments, the promoter can comprise about 1, about 2, about 3, or about 4 units each comprising, from 5′ to 3′, SEQ ID NO: 13 and SEQ ID NO: 15. In some embodiments, the promoter can comprise about 1, about 2, about 3, or about 4 units each comprising, from 5′ to 3′, SEQ ID NO: 15 and SEQ ID NO: 13. In some embodiments, the promoter can comprise about 1, about 2, about 3, or about 4 units each comprising, from 5′ to 3′, SEQ ID NO: 14 and SEQ ID NO: 12. In some embodiments, the promoter can comprise about 1, about 2, about 3, or about 4 units each comprising, from 5′ to 3′, SEQ ID NO: 12 and SEQ ID NO: 14. In some embodiments, the promoter can comprise about 1, about 2, about 3, or about 4 units each comprising, from 5′ to 3′, SEQ ID NO: 14 and SEQ ID NO: 13. In some embodiments, the promoter can comprise about 1, about 2, about 3, or about 4 units each comprising, from 5′ to 3′, SEQ ID NO: 13 and SEQ ID NO: 14. In some embodiments, the promoter can comprise about 1, about 2, about 3, or about 4 units each comprising, from 5′ to 3′, SEQ ID NO: 14 and SEQ ID NO: 14. In some embodiments, the promoter can comprise about 1, about 2, about 3, or about 4 units each comprising, from 5′ to 3′, SEQ ID NO: 14 and SEQ ID NO: 15. In some embodiments, the promoter can comprise about 1, about 2, about 3, or about 4 units each comprising, from 5′ to 3′, SEQ ID NO: 15 and SEQ ID NO: 14. In some embodiments, the promoter can comprise about 1, about 2, about 3, or about 4 units each comprising, from 5′ to 3′, SEQ ID NO: 15 and SEQ ID NO: 12. In some embodiments, the promoter can comprise about 1, about 2, about 3, or about 4 units each comprising, from 5′ to 3′, SEQ ID NO: 12 and SEQ ID NO: 15. In some embodiments, the promoter can comprise about 1, about 2, about 3, or about 4 units each comprising, from 5′ to 3′, SEQ ID NO: 15 and SEQ ID NO: 13. In some embodiments, the promoter can comprise about 1, about 2, about 3, or about 4 units each comprising, from 5′ to 3′, SEQ ID NO: 13 and SEQ ID NO: 15. In some embodiments, the promoter can comprise about 1, about 2, about 3, or about 4 units each comprising, from 5′ to 3′, SEQ ID NO: 15 and SEQ ID NO: 14. In some embodiments, the promoter can comprise about 1, about 2, about 3, or about 4 units each comprising, from 5′ to 3′, SEQ ID NO: 14 and SEQ ID NO: 15. In some embodiments, the promoter can comprise about 1, about 2, about 3, or about 4 units each comprising, from 5′ to 3′, SEQ ID NO: 15 and SEQ ID NO: 15.

### B. Nucleic Acids that Include a Promoter

This disclosure also relates to nucleic acids that include a synthetic promoter comprising a first nucleotide sequence, including synthetic promoters further comprising a second nucleotide sequence, and/or a synthetic promoter comprising a cone-specific promoter sequence, as described herein. The nucleic acids typically are designed for expression of desired protein and/or nucleic acid (typically encoded by a transgene in the nucleic acid) in cone photoreceptors. The nucleic acids can be in the form, for example, of a vector, such as an AAV vector, a lentiviral vector or other suitable vector. Accordingly, the nucleic acids will include a hybrid promoter as described herein and a transgene that encodes a desired protein or nucleic acid. The nucleic acids also will typically include regulatory elements that are well-known in the art and selected based on the desired level of expression, delivery vector and other consideration. For example, the transgene is typically operably linked to a poly A signal. Exemplary and nonlimiting examples of regulatory elements are briefly discussed herein.

### a. Posttranscription Regulatory Element

An exemplary regulatory element is a post transcriptional regulatory element, such as the Woodchuck Hepatitis Virus Posttranscriptional Regulatory element (WPRE). Regulatory elements are nucleic acid sequences that contribute to regulation of expression of a DNA sequence within which the regulatory element is located. A regulatory element may include, in some instances, three components (alpha, beta, and gamma). The activity of the regulatory element may depend on how many of the components are present.

As disclosed and exemplified herein, a WPRE element in combination with a promoter of this disclosure can result in high levels of expression of a heterologous polypeptide or a heterologous RNA (e.g., encoded by a transgene) in human cone cells.

The regulatory element can be operably linked to a nucleotide encoding a heterologous polypeptide or a heterologous RNA and other expression control elements, e.g. the promoter and a polyadenylation (Poly A) signal.

Any suitable regulatory element may be used, such as naturally occurring WPRE or a WPRE that comprises one or more mutation in the X region. For instance, a suitable WPRE with mutation in the X region is disclosed in U.S. Pat. No. 7,419,829 and presented herein as SEQ ID NO: 8.

A suitable WPRE has been described in US Publication No. US 2021/0032656. Generally, the WPRE can have a nucleotide sequence comprising SEQ ID NO: 7. The WPRE may comprise a nucleic acid sequence having at least 70% sequence identity to SEQ ID NO: 7. The WPRE can have a nucleotide sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or greater sequence identity to SEQ ID NO: 7.

The WPRE may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions. The nucleic acid substitution can be a conservative substitution or a non-conservative substitution, but preferably is a conservative substitution.

### b. Polyadenylation Signal

The nucleic acid can also include a nucleotide sequence that encodes a suitable PolyA signal. Desirably, the nucleotide sequence encoding the poly A is operably linked with the nucleotide sequence encoding the desired protein or nucleic acid for expression. Any suitable Poly A signal can be used, such as, an SV40 Poly A signal, rabbit beta-globin Poly A signal, human growth hormone (hGH) PolyA signal, bovine growth hormone Poly A signal, and the like. Human growth hormone (hGH) polyA (SEQ ID NO: 9) is a preferred PolyA.

The Poly A can be in any suitable orientation, preferably the PolyA is 3′ of any regulatory element.

The PolyA can have a nucleotide sequence comprising SEQ ID NO: 9. The Poly A may comprise a nucleic acid sequence having at least 70% sequence identity to SEQ ID NO: 9. The PolyA can have a nucleotide sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or greater sequence identity to SEQ ID NO: 9.

The Poly A may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions. The nucleic acid substitution can be a conservative substitution or a non-conservative substitution, but preferably is a conservative substitution.

### c. Packaging Sequences

The nucleic acid can further comprise a suitable packaging sequence or signal for packaging into a viral vector, such as an AAV or lentiviral vector. For example, the nucleic acid can include an inverted terminal repeat ITR, such as an AAV ITR. This can be especially appropriate if the nucleic acid is to be incorporated into an AAV vector. Typically two ITRs are included in the nucleic acid, one at the 5′ end and one at the 3′ end. For example, the nucleic acid can comprise an AAV ITR 5′ of the promoter and an AAV ITR at the 3′ terminus. Particular examples of AAV ITRs are SEQ ID NO: 10 and SEQ ID NO: 11.

ITRs may be independently selected from wild-type ITRs and optionally self-complementary (scAAV) ITRs. Other sequences (e.g. parvovirus terminal repeats) which are functionally equivalent to AAV 5′ ITRs and/or AAV 3′ ITRs can also be used.

### C. Transgene

The nucleic acid can comprise a nucleotide sequence encoding a desired polypeptide or nucleic acid. Typically the polypeptide is heterologous to the synthetic promoter, meaning that the first nucleotide sequence, the second nucleotide sequence (in embodiments wherein the synthetic promoter comprises a second nucleotide sequence), a cone-specific promoter sequence, and/or any other promoter sequences that can be included in the synthetic promoter do not drive expression of the polypeptide in nature. The synthetic promoter and the nucleotide sequence encoding the polypeptide are operably linked. The nucleotide can encode any heterologous polypeptide for which expression in cone cells is desired.

The heterologous polypeptide can be a therapeutic polypeptide, reporter protein, or optogenetic actuator, such as those described below.

In some embodiments, the nucleotide sequence encodes a therapeutic polypeptide, by which is meant the nucleotide sequence can rescue a missing or mutated gene known to cause retinal disease. Examples of retinal disease caused by missing or mutated genes, and thus can rescued by the nucleotide sequence encoding a therapeutic polypeptide, include, but are not limited to, MT-ND4 (Gene ID: 4538), MT-NDI (Gene ID: 4535), MT-ND6 (Gene ID: 4541), MT-CYB (Gene ID: 4519), MT-C03 (Gene ID: 4514), MT-ND5 (Gene ID: 4540), MT-ND2 (Gene ID: 4536), MT-COI (Gene ID: 4512), MT-ATP6 (Gene ID: 4508), MT-ND4L (Gene ID: 4539), OPA1 (Gene ID: 4976), OPA3 (Gene ID: 80207), OPA7 (Gene ID: 84233), AC02 and (Gene ID: 50). The nucleotide sequence can encode neurotrophic factors such as GDNF (Gene ID: 2668), CNTF (Gene ID: 1270), FGF2 (Gene ID: 2247), BDNF (Gene ID: 627) and EPO (Gene ID: 2056), anti-apoptotic genes such as BCL2 (Gene ID: 596) and BCL2L1 (Gene ID: 598), anti-angiogenic factors such as endostatin, angiostatin and sFlt, anti-inflammatory factors such as IL10 (Gene ID: 3586), IL1R1 (Gene ID: 3554), TGFBI (Gene ID: 7045) and IL4 (Gene ID: 3565), or the rod-derived cone viability factor (RdCVF) (Gene ID: 115861).

A signal peptide can be added to therapeutic polypeptides, in particular in to direct their import by certain organelles (such as mitochondria), to secrete them from the cell, or to insert them into the cellular membrane.

In another embodiment, the heterologous polypeptide can be an optogenetic actuator, which is a photochemically reactive polypeptide that uses vitamin A or isoforms thereof as its chromophore. An optogenetic actuator in particular is a light-gated ion pump or channel that absorbs light and is activated by light. The optogenetic actuator can be from a prokaryotic organism or a eukaryotic organism. In particular, it can be a microbial opsin or a vertebrate opsin. The optogenetic actuator can be an optogenetic activator or an optogenetic inhibitor.

An optogenetic activator causes a cell to depolarize upon exposure to light. Examples of optogenetic activators include rhodopsins, photopsins, melanopsins, pinopsins, parapinopsins, VA opsins, peropsins, neuropsins, encephalopsins, retinochromes, RGR opsins, microbial opsins with red-shifted spectral properties such as ReaChR, Chrimson or ChrimsonR, vertebrate opsins that can recruit Gi/0-signalling such as short wavelength vertebrate opsin or long wavelength vertebrate opsin, channelrhodopsins from microalgae of the genus Chlamydomonas, such as channelrhodopsin-1, channelrhodopsin-2 (from Chlamydomonas reinhardtii), and optimized or functionally improved variants (e.g. codon optimized variants, mutants, chimeras) thereof.

In some embodiments, the optogenetic actuator can be an optogenetic activator, preferably selected from channelrhodopsins, ChrimsonR and variants thereof, such as from hChR2 (L132C)-hCatCh and ChrimsonR-tdTomato.

In some embodiments, the optogenetic actuator is an optogenetic activator, preferably selected from channelrhodopsins and variants thereof, such as hChR2 (L132C)-hCatCh.

An optogenetic inhibitor causes a cell to hyperpolarize upon exposure to light. Examples of optogenetic inhibitors include, but are not limited to, halorhodopsins such as halorhodopsin (NpHR), enhanced halorhodopsins (eNpHR2.0 and eNpHR3.0) and the red-shifted halorhodopsin Halo57, archaerhodopsin-3 (AR-3), archaerhodopsin (Arch), bacteriorhodopsins such as enhanced bacteriorhodopsin (eBR), proteorhodopsins, xanthorhodopsins, Leptosphaeria maculans fungal opsins (Mac), the cruxhalorhodopsin Jaws, and optimized or functionally improved variants (e.g. codon optimized variants, mutants, chimeras) thereof.

In some embodiments, the nucleic acid operably linked to the promoter of the invention can encode a reporter protein. Preferably, the reporter protein can be detectable in living cone photoreceptor cells. The expression of a reporter protein under the control of a promoter of the disclosure allows specific detection or identification of cone photoreceptor cells. The reporter protein can be, for example, a fluorescent protein (e.g., GFP), calcium indicator (e.g. GCamP), luciferase, alkaline phosphatase, beta-galactosidase, beta-lactamase, horseradish peroxidase, and variants thereof.

In some embodiments, the reporter protein can be selected from the group consisting of fluorescent proteins, calcium indicators, alkaline phosphatases, beta-galactosidases, beta-lactamases, horseradish peroxidase, and variants thereof.

The nucleic acid can comprise a nucleotide sequence encoding an RNA heterologous to the synthetic promoter, meaning that the first nucleotide sequence, the second nucleotide sequence (in embodiments wherein the synthetic promoter comprises a second nucleotide sequence), a cone-specific promoter sequence, and/or any other promoter sequences that can be included in the synthetic promoter do not drive expression of the RNA in nature. The synthetic promoter and the nucleotide sequence encoding the RNA are operably linked. The nucleotide can encode any heterologous RNA for which expression in cone cells is desired.

In some embodiments, the heterologous RNA can be a therapeutic RNA. The RNA can be, for example, an siRNA, an shRNA, an RNAi, a miRNA, an antisense RNA, or a ribozyme. In some particular embodiments, the nucleotide sequence encodes a heterologous RNA that, when transcribed from the nucleotide sequence operably linked to the promoter, can treat or prevent an ocular disease by interfering with translation or transcription of an abnormal or excess protein associated with said disorder.

The nucleic acids described herein, or any component thereof (e.g., promoter, sequence encoding depolarizing optogenetic protein), can be optimized by sequence variation using well-known methods, for example, to achieve desired levels of expression, to reduce immunogenicity, or for other purposes. Suitable methods for optimizing nucleic acid construct by sequence alteration, including to increase expression, packaging and/or to decrease immunogenicity, for example, are well-known in the art and such modifications of the nucleic acids disclosed herein are considered to be variants of the particular nucleic acids. For example, the nucleic acids described herein, or any component thereof, can be codon optimized, CpG-depleted (See, e.g., U.S. Pat. No. 11,015,210; Y. A. Medvedeva, et al, Bioinformatics-Trends and Methodologies, 449-472 (2011)), modified to remove repeat and hairpin sequences, modified to eliminate alternative reading frames, modified to remove unwanted splice donor and acceptor sites, modified to add stuffer sequence, modified to include a dsRNA or gRNA sequence (See, e.g., Domenger and Grimm, Human Molecular Genetics, 2019, 28:R1-R12), modified to include an inducible control system (e.g. Tet on/off system) (See, e.g., Gossen et al, Science, 268:1766-1769 (1995); Harvey et al, Curr. Opin. Chem. Biol., 2:512-518 (1998)), or modified to include an ITR deleted of the terminal resolution site (trs) sequence to generate a scAAV (See, e.g., McCarty et al., Gene Therapy, 2001, 16:1248-54)). Methods for optimizing the nucleic acids disclosed herein are conventional and well-known to those of ordinary skill in the art.

### D. Vectors

The disclosure further relates to recombinant vectors comprising the nucleic acid disclosed herein or a host cell comprising the vector. An AAV vector can be based on a viral genome with the capsid and other structural proteins removed. The vectors provided herein can be suitable for gene therapy, and in particular can be suitable for targeting human cone cells. As disclosed herein, the nucleic acid comprises a synthetic promoter, and can comprise a nucleotide sequence encoding a heterologous polypeptide or RNA, a regulatory element, and a suitable polyA signal. Each of the nucleotide sequences are operable linked.

In addition, the vector may comprise additional elements for the expression of the nucleic acid. For instance, the vector may comprise one or more ITRs, a ribosome binding element, a terminator, an enhancer, a selection marker, an intron, a polyA signal, and/or an origin of replication.

In some embodiments, the AAV vector is a single stranded AAV (ssAAV). In some embodiments, the AAV vector is a self-complementary AAV (scAAV).

Many different viral and non-viral vectors and methods of their delivery are known to those of skill in the art, such as adenovirus vectors, AAV vectors, retrovirus vectors, lentiviral vectors, herpes virus vectors, liposomes, naked DNA administration and the like. See, e.g., Wright (1997), Br. J. Ophthalmol., 8(1):620-622. Numerous suitable vectors are commercially available. Such vectors typically include polyadenylation signals, etc. in conjunction with multiple cloning sites, as well as additional elements such as origins of replication, selectable marker genes (e. g., LEU2, URA3, TRP 1, HIS3, GFP), centromeric sequences, etc.

The vector suitable for the nucleic acid disclosed herein can be a viral vector, such as vectors derived from Moloney murine leukemia virus vectors (MoMLV), MSCV, SFFV, MPSV or SNV, lentiviral vectors (e.g. derived from human immunodeficiency virus (HIV), simian immunodeficiency virus (SIV), feline immunodeficiency virus (FIV), bovine immunodeficiency virus (BIV) or equine infectious anemia virus (EIAV)), adenoviral (Ad) vectors, AAV vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, and Rous sarcoma virus vectors.

The vector can be a retroviral vector, such as a lentiviral vector or a non-pathogenic parvovirus.

In some particular embodiments, the vector can be an AAV viral vector that comprises an AAV capsid. Without being bound by theory, the AAV capsid can improve selective delivery of the nucleic acid to cone cells and may also improve expression of the heterologous polypeptide or RNA.

Many suitable AAV capsids and viral backbones are well-known in the art and multiple AAV capsid serotypes are known and may be suitable for the optogenetic constructs disclosed herein. At least sixteen serotypes of AAV have been described in literature, and are referred to as AAV1, AAV2, AAV3, AAV4, AAV5, AAV6, AAV7, AAV8, AAV9, AAV10, AAV11, AAV 12, AAV13, AAV 14, AAV15, and AAV16. Many engineered and variant capsids are also well-known in the art.

Exemplary AAV capsids include, but are not limited to, AAV8-BP2, AAV-PHP.B, AAV-PHP.eB, AAV5, or AAV-NHP26. Preferred AAV capsid proteins are AAV-PHP.eB, AAV8-BP2, or AAV5.

Alternatively, to using AAV natural serotypes, artificial AAV serotypes may be used, such as AAV with a non-naturally occurring capsid. Such an artificial capsid may be generated by any suitable technique, using a selected AAV sequence (e.g., a fragment of a VP1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid or a mutated AAV capsid. A chimeric capsid comprises VP capsid proteins derived from at least two different AAV serotypes or comprises at least one chimeric VP protein combining VP protein regions or domains derived from at least two AAV serotypes.

AAV capsid proteins may also be mutated, in particular to enhance transduction efficiency. Mutated AAV capsids may be obtained from capsid modifications inserted by error prone PCR and/or peptide insertion or by including one or several amino acids substitutions. In particular, mutations may be made in any one or more of tyrosine residues of natural or non-natural capsid proteins (e.g. VP1, VP2, or VP3). Mutated residues may be surface exposed tyrosine residues. Exemplary mutations include, but are not limited to tyrosine-to-phenylalanine substitutions such as Y252F, Y272F, Y444F, Y500F, Y700F, Y704F, Y730F, Y275F, Y281F, Y508F, Y576F, Y612G, Y673F and Y720F.

The vector may be in any form, including, but not limited to, viral particles, such as rAAV particles that include a nucleic acid encoding a depolarizing optogenetic protein as described herein. If desired, the nucleic acids encoding a depolarizing optogenetic protein as described herein can be combined with other suitable nucleic acid delivery agents, for example, complexed with lipids, encapsulated within liposomes, for delivery.

The nucleic acid encoding a depolarizing optogenetic protein disclosed herein may be packaged into a virus capsid to generate a viral particle, preferably an AAV particle.

The viral particle is capable of transducing up to about 10% of primary human cone cells. For instance, the viral particle can be capable of transducing about 10%, about 15%, about 20%, about 25%, about 30%, about 35%, about 40%, about 45%, about 50%, about 55% about 60%, about 65%, about 70%, about 75%, about 80%, about 85%, about 90%, about 91%, about 92%, about 93%, about 94%, about 95%, about 96%, about 97%, about 98%, about 99%, about 100% of the primary human cone cells.

Also provided herein are production methods and cultures for the production of virus particles. The culture can comprise suitable host cells, including, for example, human-derived cell lines such as HeLa, A549, or HEK293 cells, suitable helper virus function, provided by wild-type or mutant adenovirus, e.g. temperature sensitive adenovirus, Herpes virus, or a plasmid construct providing helper functions; AAV rep and cap genes and gene products, the nucleic acid disclosed herein or a vector comprising the nucleic acid, and suitable media and media components to support viral particle production that are well-known in the art.

Suitable host cells can include, but are not limited to, mammalian cells, insect cells, plant cells, microorganisms and yeast. Host cells can also be packaging cells. Exemplary packaging and producer cells are derived from HEK293, A549 or HeLa cells.

The host cell disclosed herein may be transformed or transfected with vector comprising the nucleic acid disclosed herein or viral particle. The host cell may be any animal cell, plant cell, bacterium cell or yeast. The vector disclosed herein may be transferred into host cells using any known technique including viral infection, and may be maintained in the host cell in an ectopic form or may be integrated into the genome.

### E. Pharmaceutical Compositions

The disclosure also relates to pharmaceutical compositions that can comprise a nucleic acid, a vector, or a host cell. The pharmaceutical composition can be administered to a subject for the purposes of restoring light-sensitivity to human cone cells, in particular human cone cells that are not activated with photostimulation so that vision in a subject can be restored. Compositions comprising the nucleic acid can be suitable for administration to a subject. The pharmaceutical composition can be supplied as a liquid solution, a suspension, an emulsion, or as solid forms suitable for dissolution or suspension in liquid prior to use.

The pharmaceutical composition can comprise a pharmaceutically acceptable carrier, that is to say, any carrier that does not interfere with the effectiveness of the biological activity of the ingredients and that is not toxic to the subject to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

Suitable carriers and their formulations are described in Remington: The Science and Practice of Pharmacy, 21st Edition, David B. Troy, ed., Lippincott Williams & Wilkins (2005). Typically, an appropriate amount of a pharmaceutically-acceptable salt is used in the formulation to render the formulation isotonic, although the formulate can be hypertonic or hypotonic if desired. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers suitable for direct delivery to the eye may be administered without undue toxicity. Pharmaceutically acceptable excipients include, but are not limited to, sorbitol, any of the various tween compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Most preferably, the composition is combined with saline, Ringer's balanced salt solution (pH 7.4), and the like.

The pharmaceutical composition may optionally comprise one or more agents that facilitate delivery of a nucleic acid or a vector to a target cell, including but not limited to, transfection reagents or components thereof, such as lipids or polymers.

The pharmaceutical composition disclosed herein can be formulated for administration to the eye, in particular by intraocular injection, e.g., by subretinal and/or intravitreal or suprachoroideal administration. For intravitreal delivery, the pharmaceutical composition disclosed herein can be injected directly into the vitreous. For subretinal delivery, the pharmaceutical composition disclosed herein can be delivered in a localized subretinal bleb between the retinal pigment epithelium (RPE) and the photoreceptor layer in a surgical procedure. This can be accomplished during pars plana vitrectomy (ppV). Subretinal administration can provide the direct access to photoreceptors and the RPE. Suprachoroideal injection can provide access to the photoreceptors from the choroideal layer. Alternatively, the pharmaceutical composition can be delivered into the anterior section of the eye, in particular into the anterior chamber. Subretinal injection is the preferred administration mode.

The amount of pharmaceutical composition to be administered may be determined by standard procedure well known by those of ordinary skill in the art. Physiological data of the patient (e.g. age, size, and weight) and type and severity of the disease being treated have to be taken into account to determine the appropriate dosage.

The pharmaceutical composition may be formulated for administration by injection, e. g., by subretinal or intravitreal injection or suprachoroideal injection. Formulations for injection may be presented in unit dosage form, e. g., in ampoules or in multi-dose containers. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e. g., sterile pyrogen-free water, before use.

The pharmaceutical composition disclosed herein may also be formulated as a depot preparation or for use in an implanted delivery system. Such long-acting formulations may be administered by implantation, for example, intraocular, or by intraocular injection. The pharmaceutical composition may also be formulated as a depot preparation for use in an implanted drug delivery system or device, particularly for repeated refill of a reservoir in the implanted drug delivery system or device. Thus the pharmaceutical composition may be formulated with suitable polymeric or hydrophobic materials (for example, as an emulsion in an acceptable oil) or ion exchange resins, or as sparingly soluble derivatives, for example, as a sparingly soluble salt.

In an embodiment, the pharmaceutical composition disclosed herein can comprise a vector or viral particle comprising the nucleic acid disclosed herein. Preferably, the vector or viral particle is an AAV vector or particle. The pharmaceutical composition may comprise host cells comprising the nucleic acid disclosed herein or the viral particle comprising the nucleic acid.

Optionally, the pharmaceutical composition comprising host cells may be frozen for storage at any temperature appropriate for storage of the cells. The pharmaceutical composition can comprise viral particles and each unit dosage comprises from 10E+8 to 10E+13 viral particles measured by polymerase chain reaction using a probe specific for the virus genome.

The pharmaceutical composition may further comprise one or several additional active compounds such as corticosteroids, antibiotics, analgesics, immunosuppressants, trophic factors, or any combinations thereof.

### F. Kits

Also disclosed herein are kits comprising the nucleic acid disclosed herein, vectors comprising the nucleic acid, viral particles comprising the nucleic acid, host cells, or a pharmaceutical composition thereof.

The kit may be in the form of a pharmaceutically acceptable solution, e. g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e. g., saline, dextrose solution, etc.), to reconstitute the complex to form a solution for injection purposes.

A kit can further comprise a needle or syringe, preferably packaged in sterile form, for injecting the complex, and/or a packaged alcohol pad. Instructions are optionally included for administration of compositions by a clinician or by the patient.

### G. Methods

The disclosure also relates to methods of expressing a heterologous polypeptide or heterologous RNA in cone photoreceptor cells, e.g., in culture, in a retinal organoid, a retina explant, or a mammalian subject. The method can comprise administering to the retinal organoid, the retina explant, or the mammalian subject a nucleic acid, a vector, a host cell, or two or more thereof as described herein. The heterologous polypeptide or RNA can be expressed in cone cells.

The methods can be used in studies of the function of the heterologous polypeptide or RNA in organoids or retina explants. The methods can be used with mammalian subjects for research, prophylaxis, or treatment of diseases or disorders of mammalian vision, such as blindness.

Exemplary methods include methods comprising administering to a subject in need thereof an effective amount of a nucleic acid comprising a transgene disclosed herein, a vector comprising the nucleic acid disclosed herein, or a pharmaceutical composition thereof, to treat retinal disease by expression of the transgene in cone cells of the retina.

The vector can be an AAV vector. The nucleic acid, vector, or pharmaceutical composition thereof can be administered by subretinal injection. The nucleic acid, vector, or pharmaceutical composition may be administered before, after, or initiation of retinal disease.

The methods disclosed herein can be useful for treating or ameliorating blindness. The methods disclosed herein can be useful for restoring vision. The methods disclosed herein can be useful for restoring sensitivity to light in a human cone photoreceptor cell. The methods disclosed herein can be suitable for treating retinal degeneration.

The methods disclosed herein can be suitable for treating any disease, disorder, or condition associated vision loss, including, retinitis pigmentosa, rod-cone dystrophy, Leber's congenital amaurosis, Usher's syndrome, Bardet-Biedl Syndrome, Best disease, retinoschisis, Stargardt disease, untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, Choroideremia, post-retinal detachment cone dysfunction, or a tapetoretinal degeneration retinal vein occlusion.

Moreover, the nucleic acid molecules, vectors, or pharmaceutical compositions can be used to manufacture medicaments and/or to treat patients having a disease, disorder, or condition associated with vision loss.

The subject can be a human, dog, cat, horse, or any animal for which a vison restoration is desired.

The nucleic acid disclosed herein can be administered to the subject in an amount sufficient to at least partially restore vision.

The nucleic acid provided herein can be administered to the subject by any suitable route, including, but not limited to, intraocular (e.g., subretinal injection, intravitreal injection, or suprachoroideal injection), oral, intradermal, intrathecal, intratumoral, intramuscular, intraperitoneal, intravenous, topical, subcutaneous, percutaneous, intranasal and inhalation routes, and via scarification (scratching through the top layers of skin, e.g., using a bifurcated needle). The preferred administration route is by intraocular administration, and more preferred is administration by subretinal injection.

The dosage of the nucleic acid may depend upon the type of composition and upon the subject's age, weight, body surface area, individual condition, the individual pharmacokinetic data, and the mode of administration.

The nucleic acid can be administered to a subject who has or is at risk of developing a condition associated with vision loss. The nucleic acid can be administered before or after the disease becomes symptomatic. For example, before or after partial or complete degeneration of cone cells. The nucleic acid can be administered before or after partial or complete vision loss.

### EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following examples, which are introduced for illustration only and not intended to be limiting.

### EXAMPLES

The following are examples of methods and compositions of the invention. It is understood that various other embodiments may be practiced, given the general description provided herein.

### Example 1. Short Promoter Sequences for Specific Expression of Genes in Cone Photoreceptors

### 1.1. Methods

### Vector Constructs and Production

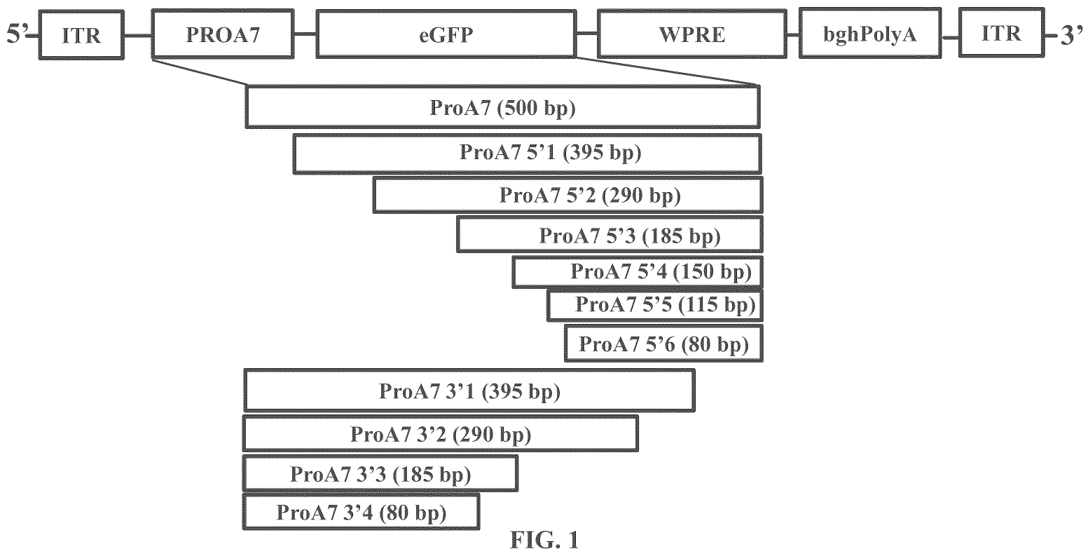

An existing cone-promoter ProA7 known to drive eGFP expression specifically in cone-photoreceptors (Jüttner et al., 2019) as well as 3′ truncated and 5′ truncated fragments thereof were each cloned into pAAV plasmid before the optimized Kozak sequence (GCCACC) and the translation start codon of eGFP coding sequence, followed by a woodchuck hepatitis virus posttranscriptional regulatory element (WPRE) and SV40 polyadenylation. (FIG. 1)

Adeno-associated viral vectors (AAV) of the serotype PHP.eB were produced in a high-throughput compatible version of the common triple transfection method as described by Grieger et al. (2006). In short: HEK293T cells were seeded in a 24 well-plate. They were transfected with the respective promoter construct together with two other plasmids (RepCap gene plasmid, Adeno virus helper gene plasmid) needed for the AAV production at equimolar ratio using jetPRIMER-Transfection Reagent and according to the manufacturer's instructions. 72 h post transfection the medium of the cells was removed, and the cells were lysed in 50 μL Buffer (150 mmol/L NaCl, 20 mmol/L tris pH 8.0) by freezing and thawing the cells 3 times.

Generation of retinal organoids from human induced pluripotent stem cells (Cowan et al., 2020)

Retinal organoids were generated as described before (Zhong et al., 2014) from human induced pluripotent stem cells (iPSCs). In brief, on day zero of differentiation, floating embryoid bodies (EBs) were generated by dissociating iPSC colonies from one well of a 6-well plate (Corning, #3516) into small colony pieces with a cell-passaging tool (Thermo Fisher Scientific, #23181010). The EBs were cultured in suspension in mTesR1 medium supplemented with 10 μmol/L blebbistatin (Sigma, #B0560-5 MG) on 3.5-cm untreated Petri dishes (Corning, #351008). On days 1 and 2, one third of the medium was exchanged with ‘neural induction medium’ (NIM) containing DMEM/F12 (GIBCO, #31331-028), 1×N2 Supplement (GIBCO, #17502-048), 1% NEAA Solution (Sigma, #M7145) and 2 μg/mL heparin (Sigma, #H3149-50KU). On day 3, to remove dead cells and debris, EBs were sedimented by gravity in a 15 mL tube, washed with NIM, and cultured in a 3.5 cm untreated Petri dish (Corning, #351008) in NIM. Half of the NIM was exchanged daily. On day 7, EBs from one 3.5-cm dish were plated onto a 6-cm dish (Corning, #430166) coated with Growth Factor-Reduced Matrigel (Corning, #356230) and then maintained with daily NIM changes.

### AAV Infection of Human Retinal Organoids

Retinal organoids were infected at week 26 with 100 μl of filtered cell culture lysate containing AAV carrying new hybrid promoter variants (serotype, AAV5) inducing the expression of enhanced GFP.

Individual organoids were placed in a single well of an ultra-low attachment U-bottom 96-well plate (Corning, #7007) and maintained at 37° C. in 5% CO2 in a mix of 15 μL culturing media and 100 μl AAV containing cell lysate. One day later, 100 μL of fresh media was added to each well. After 24 hours, and every 48 hours thereafter the solution was completely exchanged with fresh media.

### Culture of AAV-Infected Retinal Organoids

Infected organoids were cultured for 4 weeks in DMEM (GIBCO, #10569-010) supplemented with 20% Ham's F12 Nutrient Mix (GIBCO, #31765-027), 10% heat-inactivated fetal bovine serum (Millipore, #es-009-b), 1% N2 Supplement (GIBCO, #17502-048), 1% NEAA Solution (Sigma, #M7145), 100 μmol/L taurine (Sigma, #T0625), and 1 μmol/L retinoic acid (Sigma, #R2625).

### Live Image Acquisition and Processing of Whole Organoids

An Olympus “IXplore Spin confocal” spinning disc microscope system was used to record GFP expression and bright field images of retinal organoids in 96-well plates using a 4× objective.

Maximum intensity projections were generated using the OLYMPUS CellSens Software.

To evaluate the GFP expression profile of different promoter variants, the following three parameters were compared per retinal organoid: the number of GFP positive cells, the average GFP intensity of all detected cells and the cell density, which was computed by dividing the number of GFP positive cells by the organoid area.

The number of cells expressing GFP is determined in the maximum intensity projection (MIP) of the GFP channel of each image stack. Initially, Gaussian filtering was applied to reduce the overall noise level. Next, local maxima in pixel intensity were detected and then filtered in three steps: The first step removed local maxima with low contrast by calculating a local ROI window and increasing its size in each iteration. Otsu thresholding was applied on the ROI window and if there were pixels at the edges, the window size was increased. Iteration was terminated and the local maximum was accepted as an object (“cell”) if there were no active pixels at the edges after Otsu thresholding. The maximum ROI window size was set to 70×70 pixel and local maxima larger than this size were ignored. This excluded large fluorescent objects.

The next step separated cells that were close to each other. First, these cases were identified by screening the diameter and the perimeter/area ratio of each object. Subsequently, binary erosion was applied to split touching objects.

In the final filtering step, the diameter and perimeter/area ratio were analyzed. Perimeter/area ratio was more tuned to circle-like objects. Objects whose diameter fell between 6 and 100 μm were retained.

For the organoid area and cell density calculation, the bright-field images were used. Darker pixels were amplified by applying a 0.05 gamma transformation. Then Otsu threshold was applied to determine the pixels belonging to the organoid area.

### Fixation of Retinal Organoids

Organoids were fixed for 4 hours at 4° C. in 4% PFA in PBS. After fixation, samples were washed 3×30 minutes with PBS and cryopreserved in 30% sucrose in PBS overnight at 4° C. Samples were stored at −80° C. until use.

### Preparation and Staining of Cryo-Sections

Cryo-sections (20-40 μmol/L) were generated using a cryostat (MICROM International, #HM560) on organoids and human retina embedded in O.C.T compound (VWR, #25608-930). Sections were mounted onto Superfrost Plus slides (Thermo Fisher Scientific, #10149870), dried for 4 to 16 hours at room temperature and stored at −80° C. until use. Photoreceptor outer segments in retinal organoids were not preserved upon OCT embedding. Therefore, for cryo-sectioning of organoids with preserved photoreceptor outer segments, organoids were embedded in 7.5% gelatin and 10% sucrose in PBS (Lancaster and Knoblich, 2014).

For immunostainings of cryo-sections, slides were first dried for 30 minutes at room temperature and then rehydrated for 5-10 minutes in PBS. Second, slides were blocked with blocking buffer which was PBS supplemented with 10% normal donkey serum (Sigma, #S30-100 ML), 1% (wt/vol) bovine serum albumin (BSA; Sigma, #05482-25G), 0.5% Triton X-100 in PBS (Sigma, #T9284-500 ML) and 0.02% sodium azide (Sigma, #S2002-25G) at room temperature for 1 h. Sections were then incubated in a humidified chamber with primary antibodies, namely rabbit anti-GFP (Invitrogen; 1:200) and mouse monoclonal anti-human cone-arrestin 7G6 (CAR; Zhang et al., 2003).

For each slide, primary antibodies were diluted in 100 μL of ‘blocking buffer B’ which was PBS supplemented with 3% normal donkey serum, 1% BSA, 0.5% Triton X-100 in PBS and 0.02% sodium azide overnight at room temperature. After washing 3×15 minutes in PBS with 0.1% TWEEN 20 (Sigma, #P9416-100 ML), slides were incubated with secondary antibodies (Thermo Fisher Scientific, donkey secondary antibodies conjugated to Alexa Fluor 488, 568 or 647) diluted 1:500 and Hoechst diluted 1:1000 in blocking buffer for two hours at room temperature in the dark. The sections underwent washes of 2×15 minutes in PBS with 0.1% Tween, one wash of 15 minutes in PBS, and were coverslipped with ProLong Gold (Thermo Fisher Scientific, #P36934). Images were acquired with a spinning disc microscope (Olympus IXplore Spin confocal spinning disc microscope system).

### 1.2. Results

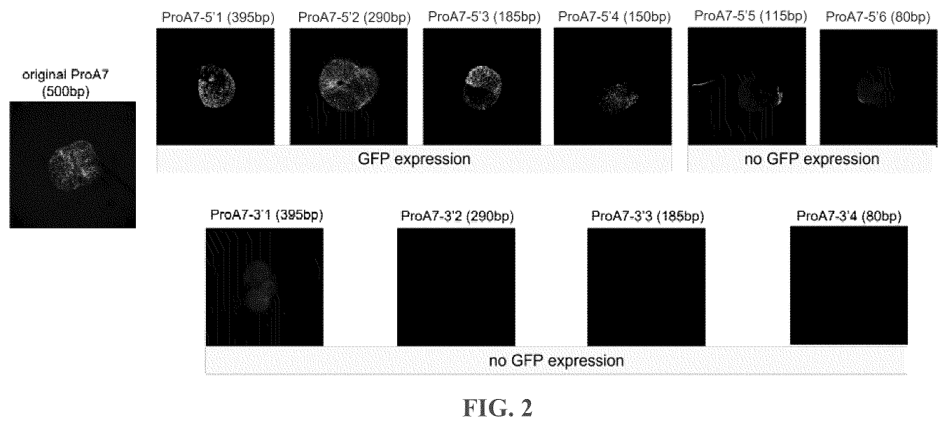

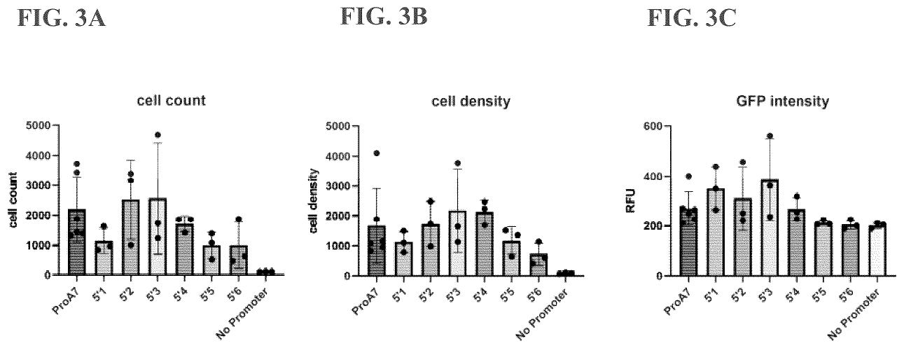

None of the 3′ truncation fragments showed any expression and thus 3′ truncation fragments were not further analyzed. Quantification of whole organoid live imaging data showed that AAVs driving GFP expression under the control of ProA7 5′1, 5′2 or 5′3 provided a comparable number of GFP expressing cells per well at similar density to full length ProA7 driven GFP (FIG. 2). The mean GFP intensity in GFP+ counted cells in organoids infected by the truncations was superior to organoids infected by AAV-ProA7-GFP (FIGS. 2-3).

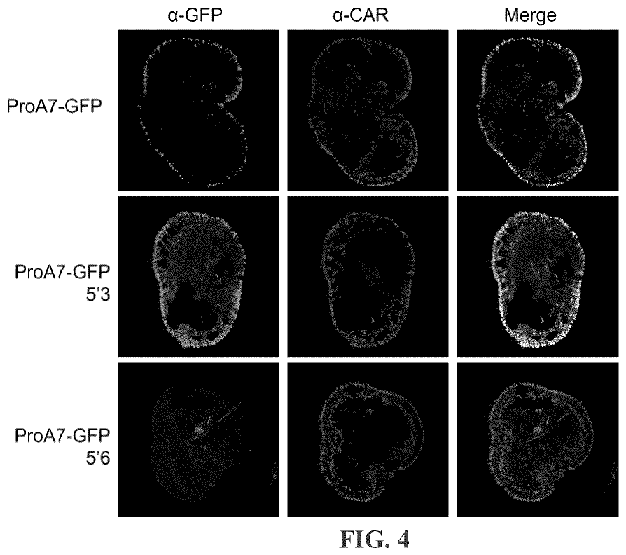

To determine which cell types express GFP, the organoids infected with ProA7 5′3 and 5′4 were further processed and analyzed. Immunohistochemistry for the specific marker of cone photoreceptors (cone arrestin, CAR) and the promoter driven GFP revealed a strong cone specific expression in organoid cryo-sections by ProA7 5′3. GFP expression was 98-100% specific for cone photoreceptors with no other cell types observed in confocal images (by analyzing at least 500 GFP positive cells each) from organoids infected with ProA7 5′3 (FIG. 4).

### Example 2. Promoter Sequences for Specific Expression of Genes in Cone Photoreceptors

### 2.1. Methods

The methods of Example 1 were followed, except as described below.

### Vector Constructs and Production

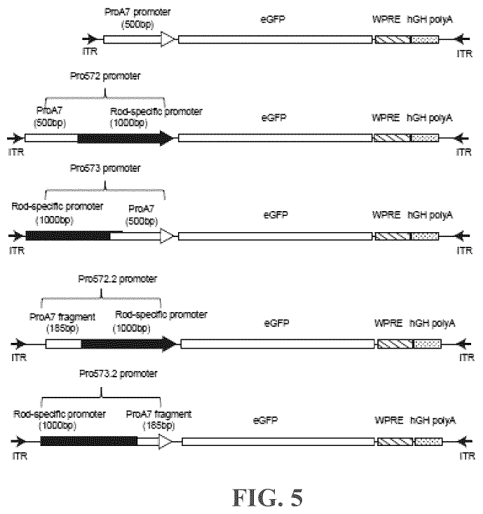

Full length and truncated 185 bp sequences of the cone specific promoter ProA7 (Jüttner et al., 2019) were fused to full length rod specific promoter ProA330 (PCT/EP2021/068653) in different orders and inserted into pAAV plasmid before the optimized Kozak sequence (GCCACC) and the translation start codon of eGFP coding sequence, followed by a woodchuck hepatitis virus posttranscriptional regulatory element (WPRE) and SV40 polyadenylation site by Gibson assembly. The vectors are depicted schematically in FIG. 5.

AAVs of serotype 5 were made as described by Grieger et al. (2006). Genome copy (GC) number titration was performed using real-time PCR (Applied Biosystems, TaqMan reagents).

AAV infection of Human Retinal Organoids

Retinal organoids were infected at week 26 with 1E+11 GC/organoid of AAV carrying new hybrid promoter variants (serotype, AAV5) inducing the expression of enhanced GFP.

Individual organoids were placed in a single well of an ultra-low attachment U-bottom 96-well plate (Corning, #7007) and maintained at 37° C. in 5% CO2 in 100 μL culturing media including 1E+11 GC AAV. Fresh media was added and exchanged as in Example 1.

### Histology Analysis

Expression density was defined as the percentage density of labeled cone photoreceptors relative to the counted total number of cone photoreceptors/mm² in cryo-sections stained with cone marker cone arrestin (CAR). All tested hybrid promoter sequences drove expression in more than 80% of CAR+ cells.

Expression specificity was quantified by determining the percentage of cone photoreceptors in the overall GFP+ cell population highlighted by the AAV. Expression in cone photoreceptor was identified by the position of the cell bodies in the retinal outer nuclear layer and the overlap with CAR marker expression.

### 2.2. Results

Quantification of whole organoid live imaging data showed AAVs driving GFP expression under the control of Pro572, Pro573, Pro572.2, or Pro573.2 provided a comparable number of GFP expressing cells per well at similar density to ProA7 driven GFP. The mean GFP intensity in GFP+ counted cells in organoids infected by AAV-{Pro572/Pro573/Pro572.2/Pro573.2}-GFP was superior to organoids infected by AAV-ProA7-GFP (FIG. 6).

To determine which cell types express GFP, the organoids infected with AAV-{Pro572/Pro573/Pro572.2/Pro573.2}-GFP were further processed and analyzed.

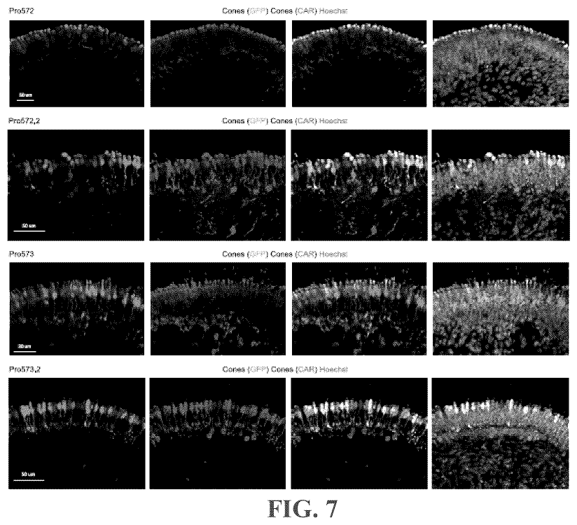

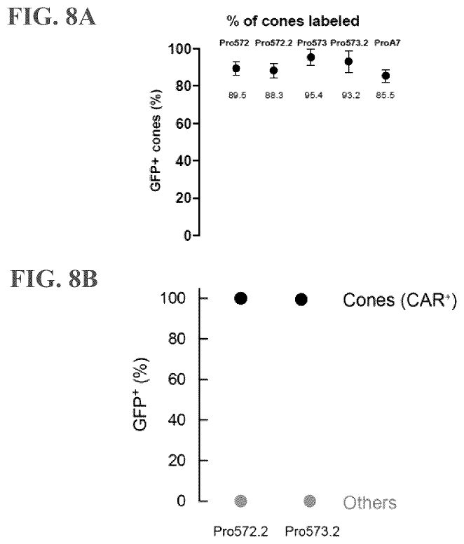

Immunohistochemistry for the marker of cone photoreceptors (cone arrestin, CAR) and the promoter driven GFP revealed a strong cone specific expression in organoid cryo-sections by Pro572, Pro573, Pro572.2, or Pro573.2 (FIG. 7). The quantitative comparison of the GFP expression density confirmed the Live imaging data with more than 85% of total cone photoreceptors expressing GFP. This is comparable to the expression density achieved by ProA7 (FIG. 8A).

GFP expression was 98-100% specific for cone photoreceptors with no other cell types observed in n=10 confocal images (by analyzing at least 500 GFP positive cells each) from organoids infected with AAV-Pro572.2-GFP and AAV-Pro573.2-GFP, respectively (FIG. 8B).

### Example 3. Efficiency of Hybrid Cone Photoreceptor Specific Promoters in Human Retina Explants

### 3.1 Methods

The methods of Example 1 were followed, except as described below.

### Vector Constructs and Production

Full length and truncated 185 bp sequences of the cone specific promoter ProA7 (Jüttner et al., 2019) were fused to full length rod specific promoter ProA330 (WO 2023/280388) in different orders and inserted into pAAV plasmid before the optimized Kozak sequence (GCCACC) and the translation start codon of eGFP coding sequence, followed by a woodchuck hepatitis virus posttranscriptional regulatory element (WPRE) and SV40 polyadenylation site by Gibson assembly. The vectors are depicted schematically in FIG. 5.

AAVs of serotype 5 and PHP.eB were made as described by Grieger et al. (2006). Genome copy (GC) number titration was performed using real-time PCR (Applied Biosystems, TaqMan reagents).

### AAV Transduction of Human Retina Cultures

Human retina cultures were maintained at 37° C. in 5% CO2 in DMEM/F-12 nutrient medium (Thermo Fisher Scientific), supplemented with 0.1% BSA, 10 μM O-acetyl-L-carnitine hydrochloride, 1 mM fumaric acid, 0.5 mM galactose, 1 mM glucose, 0.5 mM glycine, 10 mM HEPES, 0.05 mM mannose, 13 mM sodium bicarbonate, 3 mM taurine, 0.1 mM putrescine dihydrochloride, 0.35 μM retinol, 0.3 μM retinyl acetate, 0.2 μM (±)-α-tocopherol, 0.5 mM ascorbic acid, 0.05 μM sodium selenite, 0.02 μM hydrocortisone, 0.02 μM progesterone, 1 μM insulin and 0.003 μM 3,3′,5′-triiodo-L-thyronine (Sigma-Aldrich). For AAV transduction experiments, 20 μL titer matched AAV (1e11 GC) were used per human retina explant. The cultures were maintained for 5 weeks before fixation and immunohistochemical staining. Experiments were done in duplicates.

### Results 3.2

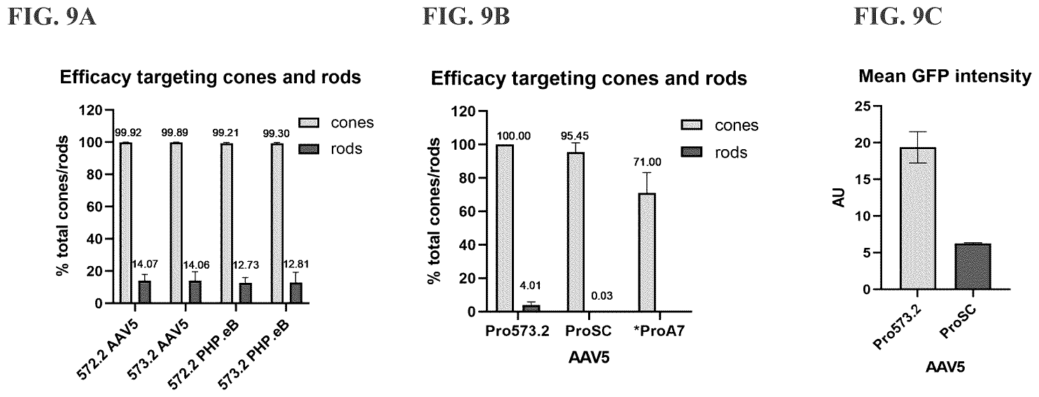

To determine whether the results obtained in retinal organoids with optimized hybrid promoters would translate to human retina, cultured human retina was transduced with AAV5 and AAVPHP.eB vector constructs expressing an enhanced GFP (eGFP) under the control of the Pro527.2, the Pro573.3, ProSC, or ProA7 promoters, and GFP expression was quantified. All constructs contained the WPRE element. Human retinas were cultured for 5 weeks prior to histochemical analysis. GFP expression with a high efficacy (more than 95%) for targeting cone photoreceptors was observed with all hybrid promoter variants (FIG. 9A). ProSC showed almost no expression in rods but was also the least effective in cone photoreceptors (FIG. 9B). AAV5-Pro573.2-EGFP-WPRE treated human retinas showed a 3-fold higher mean intensity of GFP signal than those treated with AAV5-ProSC-EGFP-WPRE (FIG. 9C). These results suggest that AAV constructs containing the optimized hybrid promoters in combination with the WPRE element express efficiently and with high specificity in retinal cone photoreceptors regardless of AAV capsid serotype, which is comparable to what was seen in transduction experiments conducted in human retinal organoids.

### Example 4. Truncated ProA330 Promoters

### 4.1 Methods

The methods of Example 1 were followed, except as described below.

### Vector Constructs and Production

An existing rod-specific promoter ProA330 (WO 2023/280388) known to drive eGFP expression specifically in rod photoreceptors as well as 3′ truncated and 5′ truncated fragments thereof were each cloned into an AAV plasmid before the optimized Kozak sequence (GCCACC) and the translation start codon of eGFP coding sequence, followed by a woodchuck hepatitis virus posttranscriptional regulatory element (WPRE). Pieces of 105 bp were cut from of the original rod-promoter ProA330 sequence (1000 bp) at either end to yield 3′1 (895 bp), 3′2 (790 bp), 3′3 (685 bp), and 3′4 (580 bp), 3′5 (475 bp), 3′6 (370 bp), 3′7 (265 bp), 3′8 (160 bp) and 3′9 (55 bp) 3′-fragments, and 5′1 (895 bp), 5′2 (790 bp), 5′3 (685 bp), and 5′4 (580 bp), 5′5 (475 bp), 5′6 (370 bp), 5′7 (265 bp), 5′8 (160 bp) and 5′9 (55 bp) 5′-fragments, respectively.

Adeno-associated viral vectors (AAV) of the serotypes AAV5 and AAVPHP.eB were produced in a high-throughput compatible version of the common triple transfection method as described by Grieger et al. (2006). The AAV were used to transduce retinal organoids and GFP expression was analyzed.

### 4.2 Results

3′ truncated and 5′ truncated fragments of Pro330 were cloned into an AAV plasmid expressing an EGFP followed by AAV vector production and testing in retinal human organoids. Whole human retinal organoid live imaging of GFP expressing cells following transduction with AAV5 and AAVPHP.eB vectors that contain varying numbers of promoter copies at a dose of 1E10 and 1E11 viral genomes (v.g) per well was performed. Vectors tested were: AAVPHP.eB-ProA7-EGFP-WPRE, AAVPHP.eB-ProSC-EGFP-WPRE, AAVPHP.eB-2×ProSC-EGFP-WPRE, AAVPHP.eB-3×ProSC-EGFP-WPRE, AAVPHP.eB-4×ProSC-EGFP-WPRE, AAV5-ProA330-EGFP-WPRE, AAVPHP.eB-2×min330-EGFP-WPRE, AAVPHP.eB-3×min330-EGFP-WPRE, AAVPHP.eB-4×min330-EGFP-WPRE, AAVPhP.eB-330-3del1del5-EGFP-WPRE, and AAVPHP.eB-3del1del6-EGFP-WPRE was performed. Negative controls include no AAV (Ctrl-noAAV) and AAV5-noPro-EGFP-WPRE (Ctrl-noP).

Quantification of whole organoid live imaging data showed that AAVs driving GFP expression under the control of Pro330 5′1, 5′2, 5′3, 5′4, 5′5, and 5′6 provided a comparable number of GFP expressing cells per well at similar density to full length Pro330 driven GFP (Table 1). The 3′ truncated ProA330 3′1 also expressed GFP at a comparable level to the ProA330 full length promoter. Since ProA330 5′6 (370 bp) and ProA330 3′1 (895 bp) still drive rod-specific GFP expression, the results suggest that the important region of ProA330 that drives cone-specific expression lies between base pairs 640-895.

TABLE 1

| Promoter | Activity | Rod specific |
|---|---|---|
| 330 5′1 (895 bp) | Yes | Yes |
| 330 5′2 (790 bp) | Yes | Yes |
| 330 5′3 (685 bp) | Yes | Yes |
| 330 5′4 (580 bp) | Yes | Yes |
| 330 5′5 (475 bp) | Yes | Yes |
| 330 5′6 (370 bp) | Maybe | Yes |
| 330 5′7 (265 bp) | No | No |
| 330 5′8 (160 bp) | No | No |
| 330 5′9 (55 bp) | Yes | No |
| 330 3′1 (895 bp) | Yes | Yes |
| 330 3′2 (790 bp) | Yes | No |
| 330 3′3 (685 bp) | No | NA (not analyzed) |
| 330 3′4 (580 bp) | No | NA |
| 330 3′5 (475 bp) | No | NA |
| 330 3′6 (370 bp) | No | NA |
| 330 3′7 (265 bp) | No | NA |
| 330 3′8 (160 bp) | No | NA |
| 330 3′9 (55 bp) | No | NA |

### Example 5. Multiple Copies of Promoters Enhance Gene Expression in Cone and Rod Photoreceptors

### 5.1 Methods

The methods of Example 1 were followed, except as described below.

### Vector Constructs and Production

Promoter replicates were designed using Geneious Prime and ordered for gene synthesis. For cone specific promoters, 1×, 2×, 3×, and 4×ProSC promoter replicates were cloned into the pAAV-SynP330-EGFP-WPRE plasmid before the optimized Kozak sequence (GCCACC) and the translation start codon of eGFP coding sequence, followed by a woodchuck hepatitis virus posttranscriptional regulatory element (WPRE). For rod-specific promoters, 1×, 2×, 3×, and 4×min330 were cloned into the pAAV-SynP330-EGFP-WPRE plasmid before the optimized Kozak sequence (GCCACC) and the translation start codon of eGFP coding sequence, followed by a woodchuck hepatitis virus posttranscriptional regulatory element (WPRE). Two deletion mutants of min330, 330-3del1del5 and 330-3del 1del6, were also cloned into the pAAV-SynP330-EGFP-WPRE plasmid before the optimized Kozak sequence (GCCACC) and the translation start codon of eGFP coding sequence, followed by a woodchuck hepatitis virus posttranscriptional regulatory element (WPRE).

AAVs of serotype 5 and PHP.eB were made as described by Grieger et al. (2006). Genome copy (GC) number titration was performed using real-time PCR (Applied Biosystems, TaqMan reagents).

### AAV Transduction of Human Retinas and Human Retinal Organoids

Human retina explants and human retinal organoids were transduced in triplicate at week 28 with a total of 1E10 and 1E11 v.g. per well of AAV containing cell lysate mixed carrying the replicate promoter variants (serotypes, AAV5 and AAVPHP.eB) inducing the expression of enhanced GFP.

Analysis of live GFP expression in whole organoids as described before.

### 5.2 Results

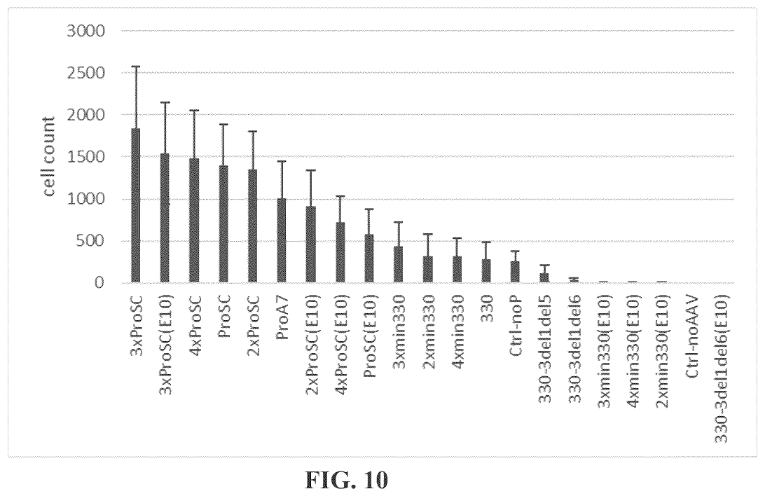

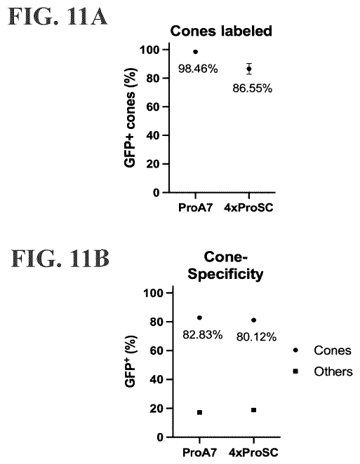

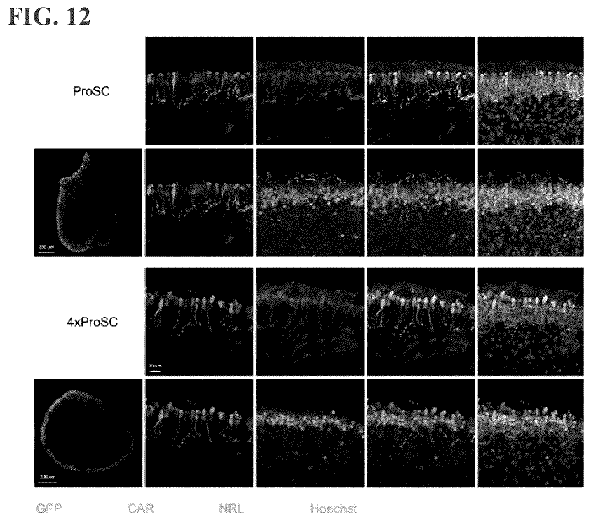

To determine the functionality of including multiple copies of rod and cone-specific promoters on gene expression, AAV vectors containing promoter replicates (2×, 3×, 4×) of the ProSC and min330 promoters driving eGFP expression were tested in organoid cultures and compared to vectors containing a single copy of the ProSC or min330 promoter. Analysis of live GFP intensity was quantified in the retinal organoids and enhanced GFP expression was observed following transduction with the AAV vectors containing the multimerized promoter constructs (FIG. 10). Variant 4×ProSC remains specific for cone photoreceptors in human retina (FIGS. 11A and 11B) and in human retinal organoids (FIG. 12). These results suggest that multiple copies of the cone and rod specific promoters, as single elements or in the context of a hybrid promoter, can enhance gene expression following AAV transduction of human retina or human retinal organoids.

### LITERATURE

Jüttner, J., Szabo, A., Gross-Scherf, B., Morikawa, R. K., Rompani, S. B., Hantz, P., & Roska, B. Targeting neuronal and glial cell types with synthetic promoter AAVs in mice, non-human primates and humans. Nature Neuroscience, 2019; 22(8):1345-1356.
Grieger J C, Choi V W, Samulski R J. Production and characterization of adeno-associated viral vectors. Nat Protoc. 2006; 1(3):1412-28.
Cowan, C. S., Renner, M., De Gennaro, M., Gross-Scherf, B., Goldblum, D., Hou, Y., & Roska, B. Cell types of the human retina and its organoids at single-cell resolution. Cell 2020; 182(6):1623-1640.
Zhong, X., Gutierrez, C., Xue, T., Hampton, C., Vergara, M. N., Cao, L.-H., Peters, A., Park, T. S., Zambidis, E. T., Meyer, J. S., et al. Generation of three-dimensional retinal tissue with functional photoreceptors from human iPSCs. Nat. Commun. 2014; 5:4047.
Zhang, H. et al. Identification and Light-Dependent Translocation of a Cone-Specific Antigen, Cone Arrestin, Recognized by Monoclonal Antibody 7G6. Invest. Ophthalmol. Vis. Sci. 44 (2003): 2858-2867.
Lancaster, M. A., and Knoblich, J. A. Generation of cerebral organoids from human pluripotent stem cells. Nat. Protoc. 2014; 9:2329-2340.

What is claimed:

1. An isolated nucleic acid, comprising a cone-specific promoter, the promoter comprising
a first nucleotide sequence of at least about 150 nucleotides which has at least 70% identity to a sequence of equal length from the sequence of SEQ ID NO: 1; and
a second nucleotide sequence of at least about 260 nucleotides having at least 70% identity to a sequence of equal length from the sequence of SEQ ID NO: 2.

2. The isolated nucleic acid of claim 1, wherein the second nucleotide sequence comprises at least about 370 nucleotides having at least 70% identity to a sequence of equal length from the sequence of SEQ ID NO:2.

3. The isolated nucleic acid of claim 1, wherein the first nucleotide sequence has at least 70% identity to a sequence of equal length from the 3′ end of the sequence of SEQ ID NO: 1.

4. The isolated nucleic acid of any one of claims 1-3, wherein the first nucleotide sequence has at least 90% identity to a sequence of equal length from the 3′ end of the sequence of SEQ ID NO: 1; the second nucleotide sequence has at least 90% identity to a sequence of equal length from the 3′ end or the 5′ end of the sequence of SEQ ID NO: 2, or both.

5. The isolated nucleic acid of any one of claims 1-4, wherein the first nucleotide sequence comprises from about 150 nucleotides to about 395 nucleotides from the 3′ end of the sequence of SEQ ID NO: 1.

6. The isolated nucleic acid of any one of claims 1-5, wherein the first nucleotide sequence comprises SEQ ID NO: 12, SEQ ID NO: 13, SEQ ID NO: 14, or SEQ ID NO: 15.

7. The isolated nucleic acid of any one of claims 1-6, wherein the second nucleotide sequence comprises from about 370 nucleotides to about 895 nucleotides from the 3′ end of SEQ ID NO: 2 or about 895 nucleotides from the 5′ end of SEQ ID NO: 2.

8. The isolated nucleic acid of any one of claims 1-7, wherein the second nucleotide sequence comprises SEQ ID NO: 21, SEQ ID NO: 22, SEQ ID NO: 23, SEQ ID NO: 24, SEQ ID NO: 25, SEQ ID NO: 26, SEQ ID NO: 30

9. The isolated nucleic acid of any one of the preceding claims, wherein the second nucleic acid is SEQ ID NO: 39.

10. The isolated nucleic acid of any one of claims 1-9, comprising from 5′ to 3′ the first nucleotide sequence and the second nucleotide sequence with no sequences therebetween.

11. The isolated nucleic acid of any one of claims 1-10, comprising from 5′ to 3′ the second nucleotide sequence and the first nucleotide sequence with no sequences therebetween.

12. The isolated nucleic acid of any one of claims 1-11, comprising two or more first nucleotide sequences, two or more second nucleotide sequences, or both.

13. The isolated nucleic acid of any one of claims 1-12, further comprising a nucleotide sequence encoding a heterologous polypeptide or a heterologous RNA, wherein the promoter and the nucleotide sequence encoding the polypeptide are operably linked.

14. The isolated nucleic acid of any one of claims 1-13, further comprising a regulatory element; wherein the promoter, the regulatory element, and the nucleotide sequence encoding the heterologous polypeptide or heterologous RNA, if present, are operably linked.

15. The isolated nucleic acid of claim 14, wherein the regulatory element is a Woodchuck Hepatitis Virus Posttranscriptional Regulatory element (WPRE).

16. The isolated nucleic acid of claim 14 or 15, further comprising a nucleotide sequence encoding a polyadenylation signal (Poly A) that is 3′ of the nucleotide sequence encoding the regulatory element; wherein the nucleotide sequence encoding the Poly A and the nucleotide sequence encoding the regulatory element are operably linked.

17. The isolated nucleic acid of any one of claims 14-16, comprising a first AAV inverted terminal repeat sequence (ITR) that is 5′ of the promoter and a second AAV ITR that is 3′ of the regulatory element and preferably 3′ of the PolyA signal.

18. The isolated nucleic acid of any one of claims 1-17, further comprising a nucleotide sequence encoding an AAV inverted terminal repeat sequence (ITR).

19. The isolated nucleic acid of any one of the preceding claims comprising one to about 10 first nucleotide sequences and/or one to about 10 second nucleotide sequences.

20. The isolated nucleic acid of any one of the preceding claims wherein the nucleic acid can contain one or more of codon optimization, CpG-reduction, alternative start site removal, repeat removal, hairpin removal, unwanted splice donor and acceptor removal, ITR terminal resolution site deletion, stuffer sequence addition, or miRNA addition.

21. An isolated nucleic acid, comprising a cone-specific promoter, the promoter comprising
a cone-specific promoter of at least about 150 nucleotides and no more than 499 nucleotides which has at least 70% identity to a sequence of equal length from the sequence of SEQ ID NO: 1.

22. The isolated nucleic acid of claim 21, comprising one to about 10 cone-specific promoters.

23. An isolated nucleic acid comprising a cone-specific promoter, wherein the promoter comprises SEQ ID NO: 40.

24. A vector comprising the isolated nucleic acid of any one of claims 1-23.

25. The vector of claim 24, further comprising a viral capsid.

26. The vector of claim 25, wherein the viral capsid is an adeno-associated viral vector (AAV) capsid selected from the group consisting of AAV8-BP2 capsid, AAV-PHP.B capsid, AAV-PHP.eB capsid, AAV5 capsid, or AAV-NHP26 capsid.

27. A host cell comprising the vector of any one of claims 24-26.

28. A kit comprising the isolated nucleic acid of any one of claims 1-23, the vector of any one of claims 24-26 the host cell of claim 27, or two or more thereof.

29. A method of expressing a heterologous polypeptide or a heterologous RNA in a retinal organoid, a retina explant, or a mammalian subject, comprising administering to the retinal organoid, the retina explant, or the mammalian subject the isolated nucleic acid of any one of claims 1-23, the vector of any one of claims 24-26, the host cell of claim 27, or two or more thereof.


SEQUENCE LISTING (40 sequences)

SEQ ID NO: 1 - DNA, 500 bp, synthetic construct
  source 1..500: mol_type = other DNA
catcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 2 - DNA, 1000 bp, synthetic construct
  source 1..1000: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 3 - DNA, 1500 bp, synthetic construct
  source 1..1500: mol_type = other DNA
catcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 4 - DNA, 1185 bp, synthetic construct
  source 1..1185: mol_type = other DNA
gttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 5 - DNA, 1500 bp, synthetic construct
  source 1..1500: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcccatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 6 - DNA, 1185 bp, synthetic construct
  source 1..1185: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 7 - DNA, 609 bp, synthetic construct
  source 1..609: mol_type = other DNA
gcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccg

SEQ ID NO: 8 - DNA, 593 bp, synthetic construct
  source 1..593: mol_type = other DNA
aatcaacctctggattacaaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaggtctgctgagactcggggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 9 - DNA, 479 bp, synthetic construct
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 10 - DNA, 130 bp, Adeno-associated virus
  source 1..130: mol_type = genomic DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 11 - DNA, 141 bp, Adeno-associated virus
  source 1..141: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 12 - DNA, 395 bp, synthetic construct
  source 1..395: mol_type = other DNA
atctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 13 - DNA, 290 bp, synthetic construct
  source 1..290: mol_type = other DNA
ggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 14 - DNA, 185 bp, synthetic construct
  source 1..185: mol_type = other DNA
gttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 15 - DNA, 150 bp, synthetic construct
  source 1..150: mol_type = other DNA
ttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 16 - DNA, 115 bp, synthetic construct
  source 1..115: mol_type = other DNA
aggggacaatccccggattaggaaggagctctccagctcgggtaaggagtctcaaggcaaggtaggcaagcaccaccggtccgcactctcgcccagcttttacgggaagaagaga

SEQ ID NO: 17 - DNA, 80 bp, synthetic construct
  source 1..80: mol_type = other DNA
gctcgggtaaggagtctcaaggcaaggtaggcaagcaccaccggtccgcactctcgcccagcttttacgggaagaagaga

SEQ ID NO: 18 - DNA, 395 bp, synthetic construct
  source 1..395: mol_type = other DNA
catcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaat

SEQ ID NO: 19 - DNA, 1724 bp, synthetic construct
  source 1..1724: mol_type = other DNA
ggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagcc

SEQ ID NO: 20 - DNA, 1782 bp, synthetic construct
  source 1..1782: mol_type = other DNA
taggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttgacaggtatttgccactaagcccagctaattgttttttatttagtagaaacggggtttcaccatgttagtcaggctggtcgggaactcctgacctcaggagatctacccgccttggcctcccaaagtgctgggattacaggcgtgtgccactgtgcccagccacttttttttagacagagtcttggtctgttgcccaggctagagttcagtggcgccatctcagctcactgcaacctccgcctcccagattcaagcgattctcctgcctcgacctcccagtagctgggattacaggtttccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccatctagagctgccgtcggggacagggctttccatagcc

SEQ ID NO: 21 - DNA, 895 bp, synthetic construct
  source 1..895: mol_type = other DNA
aaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 22 - DNA, 790 bp, synthetic construct
  source 1..790: mol_type = other DNA
acagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 23 - DNA, 685 bp, synthetic construct
  source 1..685: mol_type = other DNA
agctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 24 - DNA, 580 bp, synthetic construct
  source 1..580: mol_type = other DNA
cccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 25 - DNA, 475 bp, synthetic construct
  source 1..475: mol_type = other DNA
ccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 26 - DNA, 370 bp, synthetic construct
  source 1..370: mol_type = other DNA
acctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 27 - DNA, 265 bp, synthetic construct
  source 1..265: mol_type = other DNA
ctagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 28 - DNA, 160 bp, synthetic construct
  source 1..160: mol_type = other DNA
aatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 29 - DNA, 55 bp, synthetic construct
  source 1..55: mol_type = other DNA
attggaaggcctccgtcccagtccttctacagagtagccagtgggattcccagcc

SEQ ID NO: 30 - DNA, 895 bp, synthetic construct
  source 1..895: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcaga

SEQ ID NO: 31 - DNA, 790 bp, synthetic construct
  source 1..790: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatct

SEQ ID NO: 32 - DNA, 685 bp, synthetic construct
  source 1..685: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggca

SEQ ID NO: 33 - DNA, 580 bp, synthetic construct
  source 1..580: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtca

SEQ ID NO: 34 - DNA, 475 bp, synthetic construct
  source 1..475: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccgg

SEQ ID NO: 35 - DNA, 370 bp, synthetic construct
  source 1..370: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccaga

SEQ ID NO: 36 - DNA, 265 bp, synthetic construct
  source 1..265: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgt

SEQ ID NO: 37 - DNA, 160 bp, synthetic construct
  source 1..160: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctc

SEQ ID NO: 38 - DNA, 55 bp, synthetic construct
  source 1..55: mol_type = other DNA
aacccaagaaattacaggctgaaaccagaaaagaacacattaaagcaccaagaga

SEQ ID NO: 39 - DNA, 265 bp, synthetic construct
  source 1..265: mol_type = other DNA
acctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcaga

SEQ ID NO: 40 - DNA, 741 bp, synthetic construct
  source 1..741: mol_type = other DNA
gttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagag